(19) United States
(12) Patent Application Publication
(10) Pub. No.: US 2024/0091210 A1
(43) Pub. Date: Mar. 21, 2024

(54) USE OF 5-[[4-[2-[5-ACETYLPYRIDIN-2-YL]ETHOXY]BENZYL]-1,3-THIAZOLIDINE-2,4-DIONE AND ITS SALTS

(71) Applicant: Minoryx Therapeutics S.L., Mataró (ES)

(72) Inventors: Marc MARTINELL PEDEMONTE, Mataró (ES); Maria Pilar PIZCUETA LALANZA, Mataró (ES); Estefania TRAVER LOPEZ, Mataró, Barcelona (ES); Ana Maria GARCÍA COLLAZO, Esplugues de Llobregat (ES); Maria Angeles PÉREZ DE LA CRUZ MORENO, Majadahonda (ES)

(73) Assignee: Minoryx Therapeutics S.L., Mataró (ES)

(21) Appl. No.: 18/460,197

(22) Filed: Sep. 1, 2023

Related U.S. Application Data
(62) Division of application No. 16/972,366, filed on Dec. 4, 2020.

(30) Foreign Application Priority Data
Jun. 6, 2018 (EP) 18382402.8

(51) Int. Cl. A61K 31/4439; A61K 9/00; A61P 1/16

(52) U.S. Cl. CPC A61K 31/4439; A61K 9/0053; A61P 1/16

(57) ABSTRACT

The present disclosure relates to a method of treating or preventing a disease or disorder selected from the group consisting of a central nervous system disorder, nonalcoholic fatty liver disease, nonalcoholic steatohepatitis, a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease by administering 5-[[4-[2-[5-acetylpyridin-2-yl]ethoxy]phenyl]methyl]-1,3-thiazolidine-2,4-dione or a salt thereof to a subject in need thereof. The disclosure also relates to 5-[[4-[2-[5-acetylpyridin-2-yl]ethoxy]phenyl]methyl]-1,3-thiazolidine-2,4-dione for use in a pharmaceutical composition or in the manufacture of a medicament for the treatment or prevention of a mitochondrial disease.

### CROSS-REFERENCE TO RELATED APPLICATION

This application claims priority to European Application No. EP18382402.8, filed on Jun. 6, 2018, the entirety of which is incorporated by reference herein.

### FIELD OF DISCLOSURE

The present disclosure relates to the use of 5-[[4-[2-[5-acetylpyridin-2-yl]ethoxy]phenyl]methyl]-1,3-thiazolidine-2,4-dione and its pharmaceutically acceptable salts in the treatment or prevention of a disease or disorder selected from the group consisting of a central nervous system disorder, nonalcoholic fatty liver disease, nonalcoholic steatohepatitis, a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease. The present disclosure also provides methods of administering a therapeutically effective amount of 5-[[4-[2-[5-(1-hydroxyethyl)pyridin-2-yl]ethoxy]phenyl]methyl]-1,3-thiazolidine-2,4-dione, or a pharmaceutically acceptable salt thereof, to a patient.

### BACKGROUND

Pioglitazone is a drug marketed for use in the treatment of diabetes mellitus type 2. Pioglitazone is a potent agonist for peroxisome proliferator-activated receptor-gamma (PPAR-γ). But pioglitazone has been associated with unwanted side effects including the potential for drug to drug interactions, cardiovascular effects, fluid retention, weight gain, and bladder cancer (See, e.g., Kus et al., PLoS ONE 6(11): e27126 (2011)). High doses and/or chronic administration of pioglitazone are therefore undesirable as high systemic exposure would be likely to result in serious side effects.

Pioglitazone is a “dirty” drug which is converted to many metabolites in vivo. The metabolic pathway of pioglitazone after oral administration has been studied in several animal species and in humans, and the metabolites have been described in the literature (See, e.g., Sohda et al., Chem. Pharm. Bull. 43(12):2168-2172 (1995) and Maeshiba et al., Arzneim.-Forsch Drug Res. 47(I):29-35 (1997). At least six metabolites have been identified, named M-I to M-VI. Among these metabolites, M-II, M-III, and M-IV show some pharmacological activity but are less active than pioglitazone in diabetic preclinical models.

In the metabolic pathway of pioglitazone, 5-[[4-[2-[5-(1-hydroxyethyl)pyridin-2-yl]ethoxy]phenyl]methyl]-1,3-thiazolidine-2,4-dione (metabolite M-IV) is formed by hydroxylation of the aliphatic methylene group in the pyridin-2-yl ring, which further undergoes oxidation to form 5-[[4-[2-[5-acetylpyridin-2-yl]ethoxy]phenyl]methyl]-1,3-thiazolidine-2,4-dione (metabolite M-III). See, e.g., Maeshida et al., supra. Both of these compounds have been describe to exhibit selective peroxisome proliferator-activated receptor gamma (PPAR-γ) agonist activity.

WO 2015/150476 A1 discloses 5-[[4-[2-[5-(1-hydroxyethyl)pyridin-2-yl]ethoxy]phenyl]methyl]-1,3-thiazolidine-2,4-dione and the pharmaceutically acceptable salts thereof, for use in the treatment of central nervous system diseases or disorders. WO 2015/150476 A1 describes that 5-[[4-[2-[5-(1-hydroxyethyl)pyridin-2-yl]ethoxy]phenyl]methyl]-1,3-thiazolidine-2,4-dione can penetrate the blood-brain-barrier (BBB). International Appl. No. PCT/IB2017/057587 discloses 5-[[4-[2-[5-(1-hydroxyethyl)pyridin-2-yl]ethoxy]phenyl]methyl]-1,3-thiazolidine-2,4-dione and the pharmaceutically acceptable salts thereof, for the treatment of nonalcoholic fatty liver disease (“NAFLD”), nonalcoholic steatohepatitis (“NASH”), and other diseases and disorders.

Central Nervous System (CNS) disorders are diseases of any component of the brain and the spinal cord. CNS disorders include disorders in which the nervous system is affected during the entire progression of the diseases such as neurodegenerative diseases (e.g., Alzheimer's disease, Huntington's chorea, Parkinson's disease, amyotrophic lateral sclerosis (ALS), degenerative ataxias such as Friedrich's ataxia, multiple sclerosis, multiple system atrophy and leukodystrophies), cerebrovascular diseases (e.g., global or local ischemia, intracerebral haemorrhage, stroke), seizures and epilepsy, viral diseases (e.g., meningitis, encephalitis), brain tumors and neuroinflammatory diseases. CNS disorders also include disorders in which the nervous system is only affected during the latest stages of the development of the disorder. These disorders comprise rare metabolic diseases such as organic acidemias or fatty acid disorders and genetic mitochondrial disorders.

A wide variety of deuterium enriched 2,4-thiazolidinediones have been described in US 2014/0275180. This document also discloses their prophetic use in the treatment of a variety of different diseases.

Mitochondria are tiny subunits present inside every cell of the human body except red blood cells. Mitochondria's main role is to transform food an oxygen that enter the cells into useful energy. Pyruvate uptake across the mitochondrial inner membrane is a central branch point in cellular energy metabolism with the ability to balance glycolysis and oxidative phosphorylation and poise catabolic an anabolic metabolism. (See, e.g., Divakaruni et al., PNAS 110(14):5422-5427 (2013)). The mitochondrial pyruvate carrier (MPC) is an inner-membrane transporter that facilitates pyruvate uptake from the cytoplasm to mitochondria. It is a central regulator of mitochondrial substrate utilization, and restrictions in mitochondrial pyruvate uptake can potentiate the use of fatty acids and a range of amino acids to fuel cellular energetics and biosynthesis. (See, e.g., Divakaruni et al., J. Cell Biol. (2017)).

The MPC contains two proteins, MPC1 and MPC2, that form a carrier complex in the inner mitochondrial membrane. MPC transports pyruvate into mitochondrial matrix that is required for pyruvate metabolism and is critical for metabolic pathways. (See, e.g., McCommis et al., Biochem. J. 466: 443-454 (2015)).

Mitochondrial diseases are a group of disorders, each of which involves a mitochondrial dysfunction. Mitochondrial diseases are chronic, genetic, and often inherited disorders that that occur when mitochondria fail to produce enough energy for the body to function properly. Mitochondrial diseases can be present at birth, but can also occur at any age. These diseases can affect the cells of the brain, nerves, muscles, kidneys, heart, liver, eyes, ears, and/or pancreas. Mitochondrial dysfunction occurs when the mitochondria do not work as well as they should due to another disease or condition. Mitochondrial disease refers to a heterogeneous group of disorders that include primary and secondary mitochondrial disorders (See e.g, Niyazov et al., Mol. Syndromol. 7:122-137 (2016)). Primary mitochondrial disorders can be due to germline mutations in mitochondrial DNA (mtDNA) and/or nuclear DNA (nDNA) genes either encoding OXPHOS (oxidative phosphorylation) proteins directly or they affect OXPHOS function by impacting production of the complex machinery needed to run the OXPHOS process. Secondary mitochondrial disorders by contrast occur in many pathologic processes not involving OXPHOS, including inherited diseases with germline mutations in non-OXPHOS genes. Secondary mitochondrial disorders can also be acquired secondary to adverse environmental effects which can cause oxidative stress. Many conditions can lead to secondary mitochondrial dysfunction including autism, Parkinson's disease, Alzheimer's disease, muscular dystrophy, Lou Gehrig's disease, diabetes and cancer.

Rosiglitazone, a thiazolidinedione, has been reported to bind to the mitochondrial pyruvate carrier (MPC) at physiologic concentrations and acutely suppress pyruvate metabolism (See, e.g., Colca et al., PLOS ONE 8(5):e61551-e61551 (2013)). Divakaruni et al. describe that thiazolidinediones are acute, specific inhibitors of MPC, referring to FIG. 3C. See, Divakaruni et al., PNAS 110(14):5424 (2013). However, although pioglitazone has been mentioned in the publication, FIG. 3C does not provide any results on MPC inhibition for pioglitazone.

### SUMMARY

The present disclosure provides a method of treating or preventing a disease or disorder selected from the group consisting of a central nervous system disorder, nonalcoholic fatty liver disease (NAFLD), nonalcoholic steatohepatitis (NASH), a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease.

It has not been previously described that 5-[[4-[2-[5-acetylpyridin-2-yl]ethoxy]phenyl]methyl]-1,3-thiazolidine-2,4-dione metabolizes to 5-[[4-[2-[5-(1-hydroxyethyl)pyridin-2-yl]ethoxy]phenyl]methyl]-1,3-thiazolidine-2,4-dione. Further, it has not been previously described that administration of 5-[[4-[2-[5-acetylpyridin-2-yl]ethoxy]phenyl]methyl]-1,3-thiazolidine-2,4-dione, or its pharmaceutically acceptable salt, could be used in the treatment or prevention of a disease or disorder selected from the group consisting of a central nervous system disorder, nonalcoholic fatty liver disease, nonalcoholic steatohepatitis, a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease.

The inventors have surprisingly found that 5-[[4-[2-[5-acetylpyridin-2-yl]ethoxy]phenyl]methyl]-1,3-thiazolidine-2,4-dione, and its pharmaceutically acceptable salts, (referred to herein as “Compound (1)”) metabolize into 5-[[4-[2-[5-(1-hydroxyethyl)pyridin-2-yl]ethoxy]phenyl]methyl]-1,3-thiazolidine-2,4-dione. This transformation in vivo has not been previously described. However, the opposite transformation, i.e., the metabolization of 5-[[4-[2-[5-(1-hydroxyethyl)pyridin-2-yl]ethoxy]phenyl]methyl]-1,3-thiazolidine-2,4-dione into 5-[[4-[2-[5-acetylpyridin-2-yl]ethoxy]phenyl]methyl]-1,3-thiazolidine-2,4-dione has been described by, e.g., Maeshida et al., supra.

In addition, WO 2015/150476 A1 describes that Compound (1) does not penetrate the BBB. Inventors thus unexpectedly found that Compound (1), and pharmaceutically acceptable salts thereof, can be effectively used for treating CNS disorders by way of its metabolite, 5-[[4-[2-[5-(1-hydroxyethyl)pyridin-2-yl]ethoxy]phenyl]methyl]-1,3-thiazolidine-2,4-dione, which penetrates the BBB,

Compound (1) has one asymmetric center whereas 5-[[4-[2-[5-(1-hydroxyethyl)pyridin-2-yl]ethoxy]phenyl]methyl]-1,3-thiazolidine-2,4-dione has two asymmetric centers. The administration of Compound (1), or a pharmaceutically acceptable salt thereof, instead of 5-[[4-[2-[5-(1-hydroxyethyl)pyridin-2-yl]ethoxy]phenyl]methyl]-1,3-thiazolidine-2,4-dione, or a pharmaceutically acceptable salt thereof, to a patient in need thereof to achieve the essentially the same exposure levels and/or therapeutic efficacy for the treatment of a disease or disorder thus offers unexpected benefits from a chemistry, manufacturing and controls (CMC) perspective.

In one aspect, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, wherein said method comprises administering Compound (1),

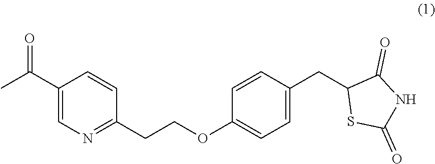 [SMILES: CC(=O)c1ccc(CCOc2ccc(CC3SC(=O)NC3=O)cc2)nc1]

or a pharmaceutically acceptable salt thereof.

In another aspect, the method provides an exposure of 5-[[4-[2-[5-(1-hydroxyethyl)pyridin-2-yl]ethoxy]phenyl]methyl]-1,3-thiazolidine-2,4-dione and 5-[[4-[2-[5-acetylpyridin-2-yl]ethoxy]phenyl]methyl]-1,3-thiazolidine-2,4-dione in the plasma at a ratio of about 7:3 (5-[[4-[2-[5-(1-hydroxyethyl)pyridin-2-yl]ethoxy]phenyl]methyl]-1,3-thiazolidine-2,4-dione: Compound (1)). Inventors have found that based on the multiple ascending dose (MAD) study upon dosing with 5-[[4-[2-[5-(1-hydroxyethyl)pyridin-2-yl]ethoxy]phenyl]methyl]-1,3-thiazolidine-2,4-dione hydrochloride, the ratio between 5-[[4-[2-[5-(1-hydroxyethyl)pyridin-2-yl]ethoxy]phenyl]methyl]-1,3-thiazolidine-2,4-dione (M-IV) and 5-[[4-[2-[5-acetylpyridin-2-yl]ethoxy]phenyl]methyl]-1,3-thiazolidine-2,4-dione (M-III) is 7:3 (M-IV:M-III).

In another aspect, the present disclosure provides a method of treating a disease or disorder in a patient in need thereof, the method comprising administering Compound (1), or a pharmaceutically acceptable salt thereof, to the patient, wherein 5-[[4-[2-[5-acetylpyridin-2-yl]ethoxy]phenyl]methyl]-1,3-thiazolidine-2,4-dione is metabolized to 5-[[4-[2-[5-(1-hydroxyethyl)pyridin-2-yl]ethoxy]phenyl]methyl]-1,3-thiazolidine-2,4-dione in the patient, and:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL;
(b) the minimum steady-state plasma drug concentration (C_{mm ss}) of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; and
the AUCₛₛ of (i), the C_{min ss} of (ii), or the AUCₛₛ and C_{min ss} of (c) is measured after at least five days of orally administering Compound (1), or a pharmaceutically acceptable salt thereof, to the patient per day.

In another aspect, the present disclosure provides a method of treating a disease or disorder in a patient in need thereof, the method comprising administering Compound (1), or a pharmaceutically acceptable salt thereof, to the patient, wherein 5-[[4-[2-[5-acetylpyridin-2-yl]ethoxy]phenyl]methyl]-1,3-thiazolidine-2,4-dione is metabolized to 5-[[4-[2-[5-(1-hydroxyethyl)pyridin-2-yl]ethoxy]phenyl]methyl]-1,3-thiazolidine-2,4-dione in the patient, and:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient is about 100 μg·h/mL to about 300 μg·h/mL;
(b) the minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient is about 2306 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 100 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 2306 to about 9126 ng/mL; and
the AUCₛₛ of (i), the C_{min ss} of (ii), or the AUCₛₛ and C_{min ss} of (c) is measured after at least five days of orally administering Compound (1), or a pharmaceutically acceptable salt thereof, to the patient per day.

In one embodiment of this aspect of the disclosure, the disease or disorder a CNS disease or disorder.

In another aspect, the present disclosure provides a method of treating or preventing a disease or disorder, wherein the method comprises administering to a subject in need thereof Compound (1), or a pharmaceutically acceptable salt thereof, in an amount effective to treat or prevent the disease or disorder, wherein the disease or disorder is selected from the group consisting of a central nervous system disorder, nonalcoholic fatty liver disease (NAFLD), nonalcoholic steatohepatitis (NASH), a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease.

In another embodiment, the disclosure provides an oral dosage form, comprising an effective amount of Compound (1), or a pharmaceutically acceptable salt thereof, the effective amount provides the following:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient of about 34 μg·h/mL to about 300 μg·h/mL;
(b) the minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient of about 55 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient of about 34 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient of about 55 to about 9126 ng/mL; and
the AUCₛₛ of (i), the C_{min ss} of (ii), or the AUCₛₛ and C_{min ss} of (c) is measured after at least five days of orally administering 5-[[4-[2-[5-acetylpyridin-2-yl]ethoxy]phenyl]methyl]-1,3-thiazolidine-2,4-dione, or a pharmaceutically acceptable salt thereof, to the patient per day. In another embodiment, the AUCₛₛ of (i), the C_{min ss} of (ii), or the AUCₛₛ and C_{min ss} of (iii) is measured after 3-15 days, e.g., 3, 4, 5, 6, 7, 8, 9, 10, 11, 12, 13, 14, or 15 days, of administering Compound (1), or a pharmaceutically acceptable salt thereof, to the patient once per day. In another embodiment, the AUCₛₛ of (i), the C_{min ss} of (ii), or the AUCₛₛ and C_{min ss} of (iii) is measured after 4 days of administering Compound (1), or a pharmaceutically acceptable salt thereof, to the patient once per day. In another embodiment, the AUCₛₛ of (i), the C_{min ss} of (ii), or the AUCₛₛ and C_{min ss} of (iii) is measured after 5 days of administering Compound (1), or a pharmaceutically acceptable salt thereof, to the patient once per day.

In another embodiment, the disclosure provides an oral dosage form, comprising an effective amount of Compound (1), or a pharmaceutically acceptable salt thereof, the effective amount provides the following:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient of about 100 μg·h/mL to about 300 μg·h/mL;
(b) the minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient of about 2306 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient of about 100 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient of about 2306 to about 9126 ng/mL; and
the AUCₛₛ of (i), the C_{min ss} of (ii), or the AUCₛₛ and C_{min ss} of (c) is measured after at least five days of orally administering 5-[[4-[2-[5-acetylpyridin-2-yl]ethoxy]phenyl]methyl]-1,3-thiazolidine-2,4-dione, or a pharmaceutically acceptable salt thereof, to the patient per day. In another embodiment, the AUCₛₛ of (i), the C_{min ss} of (ii), or the AUCₛₛ and C_{min ss} of (iii) is measured after 3-15 days, e.g., 3, 4, 5, 6, 7, 8, 9, 10, 11, 12, 13, 14, or 15 days, of administering Compound (1), or a pharmaceutically acceptable salt thereof, to the patient once per day. In another embodiment, the AUCₛₛ of (i), the C_{min ss} of (ii), or the AUCₛₛ and C_{min ss} of (iii) is measured after 4 days of administering Compound (1), or a pharmaceutically acceptable salt thereof, to the patient once per day. In another embodiment, the AUCₛₛ of (i), the C_{min ss} of (ii), or the AUCₛₛ and C_{min ss} of (iii) is measured after 5 days of administering Compound (1), or a pharmaceutically acceptable salt thereof, to the patient once per day.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

### BRIEF DESCRIPTION OF THE DRAWINGS

FIG. 1 represents the plasma levels of 5-[[4-[2-[5-acetylpyridin-2-yl]ethoxy]phenyl]methyl]-1,3-thiazolidine-2,4-dione (shown as squares) and 5-[[4-[2-[5-(1-hydroxyethyl)pyridin-2-yl]ethoxy]phenyl]methyl]-1,3-thiazolidine-2,4-dione (shown as circles) after oral administration of 50 mg/kg of 5-[[4-[2-[5-acetylpyridin-2-yl]ethoxy]phenyl]methyl]-1,3-thiazolidine-2,4-dione to mice.

FIG. 2 represents percentage of systemic exposure (AUC) of 5-[[4-[2-[5-acetylpyridin-2-yl]ethoxy]phenyl]methyl]-1,3-thiazolidine-2,4-dione (labelled M3 in the figure) and 5-[[4-[2-[5-(1-hydroxyethyl)pyridin-2-yl]ethoxy]phenyl]methyl]-1,3-thiazolidine-2,4-dione (labeled MIN-102 in the figure) following a single oral dose administration of either of MIN-102 or M3 to male mice (Dose: 4.5 mg/kg).

FIG. 3 represents percentage of systemic exposure (AUC) of 5-[[4-[2-[5-acetylpyridin-2-yl]ethoxy]phenyl]methyl]-1,3-thiazolidine-2,4-dione (labelled M3 in the figure) and 5-[[4-[2-[5-(1-hydroxyethyl)pyridin-2-yl]ethoxy]phenyl]methyl]-1,3-thiazolidine-2,4-dione (labeled MIN-102 in the figure) following a single oral dose administration of either of MIN-102 or M-III to male rats (Dose: 10 mg/kg).

FIG. 4 is a line graph showing the relationship of trough value at steady state (labeled Cₘᵢₙ ng/mL in the figure) to area under the curve at steady state (labeled AUC ng·h/mL in the figure) following daily oral dosing 135 mg and 270 mg of 5-[[4-[2-[5-(1-hydroxyethyl)pyridin-2-yl]ethoxy]phenyl]methyl]-1,3-thiazolidine-2,4-dione hydrochloride to human patients.

FIG. 5 represents a comparison of the MPC inhibitory effects of MIN-102 and pioglitazone in an in vitro MPC inhibitory activity model using BRET-assay in HEK cells.

FIG. 6A represents the effect of MIN-102 on OCR in Hela cells.

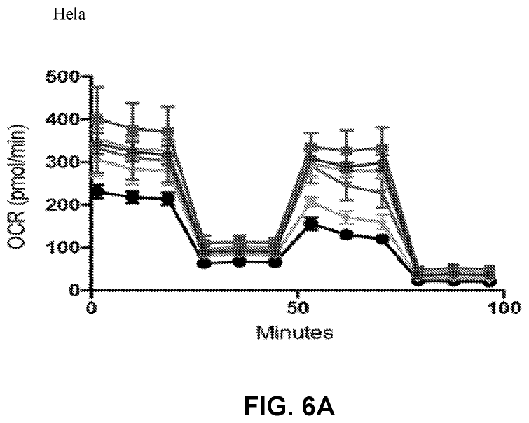

FIG. 6B represents the effect of MIN-102 on OCR in A549 cells.

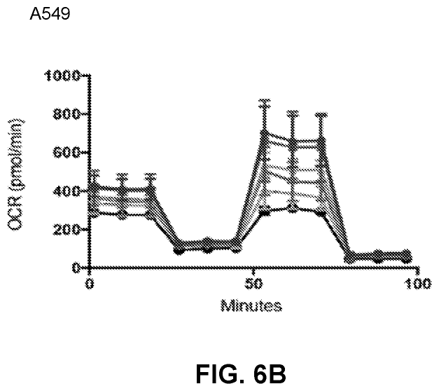

FIG. 7A represents the effect of MIN-102 on OCR in wild type MDS MB231 cells.

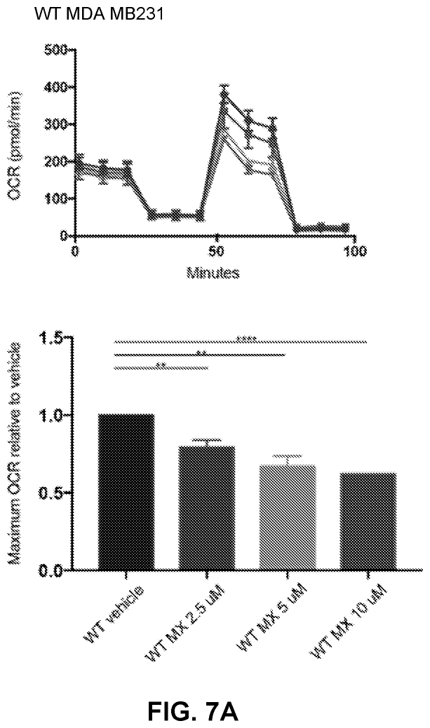

FIG. 7B represents the effect of MIN-102 on OCR in MDS MB231 KO cells.

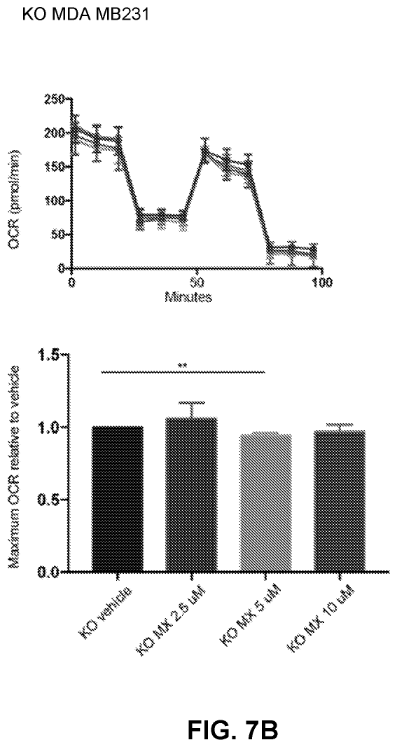

FIG. 8 represents a comparison of adiponectin levels in Sprague Dawley rats after treatment with MIN-102.

FIG. 9 depicts percentages of systemic exposure (AUC) of 5-[[4-[2-[5-acetylpyridin-2-yl]ethoxy]phenyl]methyl]-1,3-thiazolidine-2,4-dione (labelled M3 in the figure) and 5-[[4-[2-[5-(1-hydroxyethyl)pyridin-2-yl]ethoxy]phenyl]methyl]-1,3-thiazolidine-2,4-dione (labeled MIN-102 in the figure) following a single oral dose administration of either 5-[[4-[2-[5-acetylpyridin-2-yl]ethoxy]phenyl]methyl]-1,3-thiazolidine-2,4-dione or MIN-102 to male dogs (dose 3 mg/kg).

FIG. 10 depicts the conversion of 5-[[4-[2-[5-acetylpyridin-2-yl]ethoxy]phenyl]methyl]-1,3-thiazolidine-2,4-dione (labeled M-III in the figure) to 5-[[4-[2-[5-(1-hydroxyethyl)pyridin-2-yl]ethoxy]phenyl]methyl]-1,3-thiazolidine-2,4-dione (labeled M-IV in the figure) in MDCKII cell culture after 120 minutes of incubation.

### DETAILED DESCRIPTION

The methods of the present disclosure comprise administering 5-[[4-[2-[5-acetylpyridin-2-yl]ethoxy]phenyl]methyl]-1,3-thiazolidine-2,4-dione, or a pharmaceutically acceptable salt thereof, to a patient. It has been unexpectedly discovered that 5-[[4-[2-[5-acetylpyridin-2-yl]ethoxy]phenyl]methyl]-1,3-thiazolidine-2,4-dione (also called as 5-{4-[2-(5-acetylpyridin-2-yl)ethoxy]benzyl}-thiazolidine-2,4-dione, ketopioglitazone, keto pioglitazone, or M-III), and pharmaceutically acceptable salts thereof, metabolizes to 5-[[4-[2-[5-(1-hydroxyethyl)pyridin-2-yl]ethoxy]phenyl]methyl]-1,3-thiazolidine-2,4-dione (also called as 5-(4-(2-(5-(1-hydroxyethyl)pyridine-2-yl)ethoxy)benzyl)thiazolidine-2,4-dione, hydroxypioglitazone, hydroxy pioglitazone, or M-IV) in a human or animal body.

5-[[4-[2-[5-acetylpyridin-2-yl]ethoxy]phenyl]methyl]-1,3-thiazolidine-2,4-dione has one chiral center in the 5-position of the thiazolidine-dione ring as shown by the arrow:

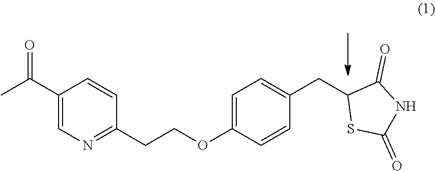 [SMILES: CC(=O)c1ccc(CCOc2ccc(CC3SC(=O)NC3=O)cc2)nc1]

As used herein, the terms “5-[[4-[2-[5-acetylpyridin-2-yl]ethoxy]phenyl]methyl]-1,3-thiazolidine-2,4-dione: or “Compound (1)” includes all possible stereoisomers, see Compounds (2) and (3) below, and mixtures thereof, including racemic mixtures of 5-[[4-[2-[5-acetylpyridin-2-yl]ethoxy]phenyl]methyl]-1,3-thiazolidine-2,4-dione.

In another embodiment, Compound (1) is (R)-5-[[4-[2-[5-acetylpyridin-2-yl]ethoxy]phenyl]methyl]-1,3-thiazolidine-2,4-dione (Compound (2)), or a pharmaceutically acceptable salt thereof.

In another embodiment, Compound (1) is (S)-5-[[4-[2-[5-acetylpyridin-2-yl]ethoxy]phenyl]methyl]-1,3-thiazolidine-2,4-dione (Compound (3)), or a pharmaceutically acceptable salt thereof.

Reference to compounds (1) to (3) in the present disclosure is intended to designate Compounds (1) to (3) having hydrogen atoms which are predominantly in the form of its isotope ¹H, i.e. no more than 1% of the total number of hydrogen atoms per mole of compound are in the form of the ²H isotope (deuterium). In one embodiment, no more than 0.015% (which is the natural abundance of deuterium) of the total number of hydrogen atoms per mole of compound are in the form of the ²H isotope (deuterium).

In one embodiment, the patient can be administered a mixture comprising a non-equimolar amount of each Compound (2) and (3), or a pharmaceutically acceptable salt thereof. In another embodiment, the mixture comprises each of Compound (2) and (3), or a pharmaceutically acceptable salt thereof, in an amount of 45%±10% w/w. In another embodiment, the mixture comprises each of Compound (2) and (3), or a pharmaceutically acceptable salt thereof, in an amount of 50%±5% w/w.

In another embodiment, the patient can be administered a mixture comprising each Compound (2) and (3), or a pharmaceutically acceptable salt thereof, wherein the mixture comprises an enantiomeric excess of one of the Compounds (2) or (3). In another embodiment, the patient can be administered a mixture comprising an equimolar amount of each Compound (2) and (3), or a pharmaceutically acceptable salt thereof, i.e., each compound in an amount of 50% w/w.

In another embodiment of the mixture of compounds (2) or (3), the two compounds mentioned are present in equimolar quantities. Said mixtures can also be enantiomerically enriched with respect to one compound (2) or (3).

Another aspect of the disclosure, suitable pharmaceutically acceptable salts of Compound (1) include, for example, pharmaceutically acceptable acid addition salts of the Compounds of the Disclosure can be prepared from the following acids, including without limitation, formic, acetic, propionic, benzoic, acetic, propionic, benzoic, succinic, glycolic, gluconic, lactic, maleic, malic, tartaric, citric, nitric, ascorbic, glucuronic, maleic, fumaric, pyruvic, aspartic, glutamic, benzoic, hydrochloric, hydrobromic, hydroiodic, isocitric, xinafoic, tartaric, trifluoroacetic, pamoic, propionic, anthranilic, mesylic, napadisylate, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. In an embodiment, the pharmaceutically acceptable salts include the salts of hydrochloric acid and hydrobromic acid. In an embodiment, the pharmaceutically acceptable salt includes the salt of the hydrochloric acid.

Compound (1) and its salts can be prepared by any suitable method known in the art, such as by the processes described in Sohda et al., Chem. Pharm. Bull. 43(12):2168-2172 (1995); Tanis et al., J. Med. Chem. 39:5053-5063 (1996); and WO 93/224454 A1. 5-[[4-[2-[5-Acetylpyridin-2-yl]ethoxy]phenyl]methyl]-1,3-thiazolidine-2,4-dione is also commercially available from, for example, Santa Cruz Biotechnology and Toronto Research Chemicals (Toronto, Ontario, Canada). 5-[[4-[2-[5-Acetylpyridin-2-yl]ethoxy]phenyl]methyl]-1,3-thiazolidine-2,4-dione can also be synthesized as described in Example 6 from 5-[[4-[2-[5-(1-hydroxyethyl)pyridin-2-yl]ethoxy]phenyl]-methyl]-1,3-thiazolidine-2,4-dione. 5-[[4-[2-[5-(1-hydroxyethyl)pyridin-2-yl]ethoxy]-phenyl]methyl]-1,3-thiazolidine-2,4-dione can be prepared, e.g., as described in WO 2015/150476 A1 or WO 2018/116281 A1. 5-[[4-[2-[5-(1-hydroxyethyl)pyridin-2-yl]ethoxy]phenyl]methyl]-1,3-thiazolidine-2,4-dione is also commercially available from, for example, Santa Cruz Biotechnology and Toronto Research Chemicals (Toronto, Ontario, Canada).

### METHODS AND USES OF THE DISCLOSURE

In one aspect, the disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, wherein said method comprises administering Compound (1), or a pharmaceutically acceptable salt thereof, to the patient.

In another embodiment, the method provides an exposure of said 5-[[4-[2-[5-(1-hydroxyethyl)pyridin-2-yl]ethoxy]phenyl]methyl]-1,3-thiazolidine-2,4-dione and Compound (1) in the plasma of the patient at a ratio of about 7:3 (5-[[4-[2-[5-(1-hydroxyethyl)pyridin-2-yl]ethoxy]phenyl]methyl]-1,3-thiazolidine-2,4-dione: Compound (1)).

In another embodiment, about 10 mg to about 500 mg of Compound (1), or a pharmaceutically acceptable salt thereof, is administered to the patient.

In another embodiment, about 100 mg to about 200 mg of Compound (1), or a pharmaceutically acceptable salt thereof, is administered to the patient.

In one aspect, the present disclosure provides a concentration control approach to administer a therapeutically effective amount of Compound (1), or a pharmaceutically acceptable salt thereof, to a patient in need thereof. This approach is based on the measured steady-state exposure, e.g., AUCₛₛ or C_{min ss}, of 5-[[4-[2-[5-(1-hydroxyethyl)pyridin-2-yl]ethoxy]phenyl]methyl]-1,3-thiazolidine-2,4-dione in plasma. Using this approach, a calculated adjustment of the initial, e.g., the first 5-14 days, dosage amount of Compound (1), or pharmaceutically acceptable salt thereof, balances the therapeutic efficacy of 5-[[4-[2-[5-(1-hydroxyethyl)pyridin-2-yl]ethoxy]phenyl]methyl]-1,3-thiazolidine-2,4-dione against toxicity and unwanted side effects to provide the maximum benefit to patients over time, e.g., weeks, months, or years. Foremost among such patient are human subjects.

In another aspect, the present disclosure provides a concentration control approach that periodically monitors Compound (1) exposure in a patient during the entire duration of treatment in the patient. Using this approach, a calculated adjustment of the dosage amount of Compound (1), or pharmaceutically acceptable salt thereof, may occur at any time, e.g., after about 4 weeks, after about 6 weeks, after about 8 weeks, after about 10 weeks, after about 12 weeks, after about 4 months, after about 6 months, after about 8 months, after about 10 months, or after about 1 year, or more, during treatment with Compound (1), or pharmaceutically acceptable salt thereof.

In another embodiment, the disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(i) administering an amount of Compound (1), or a pharmaceutically acceptable salt thereof, to the patient per day;
(ii) obtaining a plasma sample from the patient after at least 5 days of administering according to (a);
(iii) determining the plasma concentration of 5-[[4-[2-[5-(1-hydroxyethyl)pyridin-2-yl]ethoxy]phenyl]methyl]-1,3-thiazolidine-2,4-dione in the plasma sample obtained in (ii); and
(iv) administering a recalculated amount of Compound (1), or a pharmaceutically acceptable salt thereof, in milligrams, to the patient per day.

In another embodiment, the recalculated amount (the “new amount in mg”) of Compound (1), or a pharmaceutically acceptable salt thereof, in milligrams, in (iv) is calculated according to the Equation 1:

$$n e w a m o u n t in mg = S D \times ( \frac{C M T}{P C} ) , \qquad Equation 1$$

wherein:
SD is the amount of Compound (1), or a pharmaceutically acceptable salt thereof, administered to the patient in (i) in mg, see above;
CMT is the C_{min target} in ng/mL of 5-[[4-[2-[5-(1-hydroxyethyl)pyridin-2-yl]ethoxy]phenyl]methyl]-1,3-thiazolidine-2,4-dione;
C_{min target}=(target AUC ng·h/mL×0.0341±20%)−1104±20%; and
PC is the plasma concentration in ng/mL of 5-[[4-[2-[5-(1-hydroxyethyl)pyridin-2-yl]ethoxy]phenyl]methyl]-1,3-thiazolidine-2,4-dione determined in (iii).

In another embodiment, the disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(i) administering an amount of Compound (1), or a pharmaceutically acceptable salt thereof, to the patient per day;
(ii) obtaining a plasma sample from the patient after 3-15 days, e.g., 3, 4, 5, 6, 7, 8, 9, 10, 11, 12, 13, or 14 days, of administering according to (a);
(iii) determining the plasma concentration of 5-[[4-[2-[5-(1-hydroxyethyl)pyridin-2-yl]ethoxy]phenyl]methyl]-1,3-thiazolidine-2,4-dione in the plasma sample obtained in (ii); and
(iv) administering a recalculated amount of Compound (1), or a pharmaceutically acceptable salt thereof, in milligrams, to the patient per day according to Equation 1.

In another embodiment, CMT or (C_{min target})=(target AUC ng·h/mL×0.0341±10%)−1104±10% in Equation 1.

In another embodiment, CMT or (C_{min target})=(target AUC ng·h/mL×0.0341±5%)−1104±5% in Equation 1.

In another embodiment, CMT or (C_{min target})=(target AUC ng·h/mL×0.0341−1104 in Equation 1.

In another embodiment, the target AUC is about 100 μg·h/mL to about 300 μg·h/mL.

In another embodiment, the target AUC is about 100 μg·h/mL to about 200 μg·h/mL.

In another embodiment, the target AUC is about 100 μg·h/mL, about 110 μg·h/mL, about 120 μg·h/mL, about 130 μg·h/mL, about 140 μg·h/mL, about 150 μg·h/mL, about 160 μg·h/mL, about 170 μg·h/mL, about 180 μg·h/mL, about 190 μg·h/mL, or about 200 μg·h/mL.

In another embodiment, the amount of Compound (1), or a pharmaceutically acceptable salt thereof, administered to the patient in (i) is about 10 mg to about 500 mg.

In another embodiment, the amount of Compound (1), or a pharmaceutically acceptable salt thereof, administered to the patient in (i) is about 50 mg to about 500 mg.

In another embodiment, the amount of Compound (1), or a pharmaceutically acceptable salt thereof, administered to the patient in (i) is about 100 mg to about 200 mg.

In another embodiment, the amount of Compound (1), or a pharmaceutically acceptable salt thereof, administered to the patient in (i) is about 100 mg, about 110 mg, about 120 mg, about 130 mg, about 140 mg, about 150 mg, about 160 mg, about 170 mg, about 180 mg, about 190 mg, or about 200 mg.

In another embodiment, the target AUC is about 200 μg·h/mL and the amount of Compound (1), or a pharmaceutically acceptable salt thereof, administered to the patient in (i) is about 150 mg.

In another embodiment, the plasma sample is obtained from the patient after at least 7 days of administering according to (i).

In another embodiment, a plasma sample is obtained from the patient after at least 10 days of administering according to (i).

In another embodiment, a plasma sample is obtained from the patient after at least 14 days of administering according to (i).

In another embodiment, the Compound (1), or a pharmaceutically acceptable salt thereof, is administered orally to the patient in (i) and (iv).

In another embodiment, Compound (1) HCl is administered to the patient per day in (i) and (iv).

In another embodiment, about 10 mg to about 100 mg of Compound (1), or a pharmaceutically acceptable salt thereof, is administered to the patient per day in (i). In another embodiment, this dosage is administered for a non-CNS disease.

In another embodiment, about 90 mg of Compound (1), or a pharmaceutically acceptable salt thereof, is administered to the patient per day in (i).

In another embodiment, about 120 mg of Compound (1), or a pharmaceutically acceptable salt thereof, is administered to the patient per day in (i).

In another embodiment, about 150 mg of Compound (1), or a pharmaceutically acceptable salt thereof, is administered to the patient per day in (i).

In another embodiment, about 180 mg of Compound (1), or a pharmaceutically acceptable salt thereof, is administered to the patient per day in (i).

In another embodiment, about 210 mg of Compound (1), or a pharmaceutically acceptable salt thereof, is administered to the patient per day in (i).

In another embodiment, about 150 mg of Compound (1) HCl is administered to the patient per day in (i).

In another embodiment, a recalculated amount of Compound (1) HCl is administered to the patient per day in (iv).

In another embodiment, the Compound (1) HCl is administered to the patient in (i) and (iv) as a suspension comprising about 5-15 mg of Compound (1) HCl per mL.

In another embodiment, the Compound (1), or pharmaceutically acceptable salt thereof, e.g., 5-[[4-[2-[5-acetylpyridin-2-yl]ethoxy]phenyl]methyl]-1,3-thiazolidine-2,4-dione hydrochloride, is administered to a patient in (i) and (iv) having a disease or disorder.

In another aspect, the disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, based on the plasma and/or cerebrospinal fluid (CSF) concentration of a biomarker, e.g., a PPAR-γ engagement biomarker, in a sample obtained from the patient, the method comprising administering 5-[[4-[2-[5-acetylpyridin-2-yl]ethoxy]phenyl]methyl]-1,3-thiazolidine-2,4-dione, or a pharmaceutically acceptable salt thereof, to the patient.

In another embodiment, the disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(a) determining the plasma or cerebrospinal fluid (CSF) concentration of a biomarker in a sample obtained from the patient;
(b) administering an amount of Compound (1), or a pharmaceutically acceptable salt thereof, to the patient per day;
(c) obtaining a plasma or CSF sample from the patient after at least 5 days of administering according to (b);
(d) determining the plasma or CSF concentration of the biomarker in the plasma or CSF sample obtained in (c); and
(e) administering a recalculated amount of Compound (1), or a pharmaceutically acceptable salt thereof, in milligrams, based on the biomarker concentration in the plasma or CSF sample obtained in (d).

In another embodiment, the biomarker is a PPAR-7 engagement biomarker in the plasma.

In another embodiment, the biomarker is a PPAR-7 engagement biomarker in the CSF.

In another embodiment, the PPAR-7 engagement biomarker in the CSF is adiponectin or FABP4, and the concentration of adiponectin and/or FABP4 increases as a result of administering Compound (1), or a pharmaceutically acceptable salt thereof, to the patient.

In another embodiment, the biomarker is an inflammatory biomarker in the CSF.

In another embodiment, the inflammatory biomarker in the CSF is IP10, IL6, IL8, or MCP-1, and the concentration of IP10, IL6, IL8, and/or MCP-1 decreases as a result of administering Compound (1), or a pharmaceutically acceptable salt thereof, to the patient.

In another aspect, the disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising determining the plasma concentration of adiponectin in a sample obtained from the patient; and
(a) administering an amount of Compound (1), or a pharmaceutically acceptable salt thereof, to the patient per day;
(b) obtaining a plasma sample from the patient after at least 5 days of administering according to (a);
(c) determining the plasma concentration of adiponectin in the plasma sample obtained in (b); and
(d) administering a recalculated amount of Compound (1), or a pharmaceutically acceptable salt thereof, in milligrams, based on the concentration of adiponectin in the plasma sample obtained in (c), wherein:
   (i) an increase in adiponectin of about 200% or less in (c) relative to (a) comprises administering a greater amount of Compound (1), or a pharmaceutically acceptable salt thereof, in mg per day, to the patient;
   (ii) an increase in adiponectin of about 600% or more in (c) relative to (a) comprises administering a lesser amount of Compound (1), or a pharmaceutically acceptable salt thereof, in mg per day, to the patient; and
   (iii) an increase in adiponectin of about 200% to about 600% in (c) relative to (a) comprises administering the same amount of Compound (1), or a pharmaceutically acceptable salt thereof, in mg per day, to the patient.

In another embodiment, the present disclosure provides methods of treating a disease or disorder in a patient in need thereof, the method comprising administering 5-[[4-[2-[5-acetylpyridin-2-yl]ethoxy]phenyl]methyl]-1,3-thiazolidine-2,4-dione (Compound (1)), or a pharmaceutically acceptable salt thereof, to the patient, wherein 5-[[4-[2-[5-acetylpyridin-2-yl]ethoxy]phenyl]methyl]-1,3-thiazolidine-2,4-dione is metabolized to 5-[[4-[2-[5-(1-hydroxyethyl)pyridin-2-yl]ethoxy]phenyl]methyl]-1,3-thiazolidine-2,4-dione in the patient, and:
(a) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL;
(b) the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; and
the AUCₛₛ of (i), the C_{min ss} of (ii), or the AUCₛₛ and C_{min ss} of (c) is measured after at least five days of administering Compound (1), or a pharmaceutically acceptable salt thereof, to the patient per day.

In another embodiment, the present disclosure provides methods of treating a disease or disorder, e.g., a CNS disease or disorder, in a patient in need thereof, the method comprising administering 5-[[4-[2-[5-acetylpyridin-2-yl]ethoxy]phenyl]methyl]-1,3-thiazolidine-2,4-dione (Compound (1)), or a pharmaceutically acceptable salt thereof, to the patient, wherein 5-[[4-[2-[5-acetylpyridin-2-yl]ethoxy]phenyl]methyl]-1,3-thiazolidine-2,4-dione is metabolized to 5-[[4-[2-[5-(1-hydroxyethyl)pyridin-2-yl]ethoxy]phenyl]methyl]-1,3-thiazolidine-2,4-dione in the patient, and:
(a) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 100 μg·h/mL to about 300 μg·h/mL;
(b) the C_{min ss} of 5-[[4-[2-[5-(1-hydroxyethyl)pyridin-2-yl]ethoxy]phenyl]methyl]-1,3-thiazolidine-2,4-dione in plasma from the patient is about 2306 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 100 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 2306 to about 9126 ng/mL; and
the AUCₛₛ of (i), the C_{min ss} of (ii), or the AUCₛₛ and C_{min ss} of (c) is measured after at least five days of administering Compound (1), or a pharmaceutically acceptable salt thereof, to the patient per day.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 100 μg·h/mL to about 300 μg·h/mL for treating a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 100 μg·h/mL to about 200 μg·h/mL for treating a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 130 μg·h/mL to about 200 μg·h/mL for treating a disease or disorder

In another embodiment, the AUCₛₛ of 5-[[4-[2-[5-(1-hydroxyethyl)pyridin-2-yl]ethoxy]phenyl]methyl]-1,3-thiazolidine-2,4-dione is about 150 μg·h/mL to about 250 μg·h/mL for treating a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 175 μg·h/mL to about 225 μg·h/mL for treating a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 100 μg·h/mL, about 110 μg·h/mL, about 120 μg·h/mL, about 130 μg·h/mL, about 140 μg·h/mL, about 150 μg·h/mL, about 160 μg·h/mL, about 170 μg·h/mL, about 180 μg·h/mL, or about about 190 μg·h/mL for treating a disease or disorder.

In another embodiment, the present disclosure provides methods of treating a disease or disorder, e.g., a non-CNS disease or disorder, e.g., NASH, in a patient in need thereof, the method comprising administering 5-[[4-[2-[5-(1-hydroxyethyl)pyridin-2-yl]ethoxy]phenyl]methyl]-1,3-thiazolidine-2,4-dione (Compound (1)), or a pharmaceutically acceptable salt thereof, to the patient, wherein:
(a) the AUCₛₛ of Compound (1) in plasma from the patient is about 30 μg·h/mL to about 300 μg·h/mL; and
(b) the AUCₛₛ is measured after administering Compound (1), or a pharmaceutically acceptable salt thereof, to the patient once per day for five or more days.

In another embodiment, the AUCₛₛ of Compound (1) in plasma from the patient is about 30 μg·h/mL to about 250 μg·h/mL.

In another embodiment, the AUCₛₛ of Compound (1) in plasma from the patient is about 30 μg·h/mL to about 200 μg·h/mL.

In another embodiment, the AUCₛₛ of Compound (1) in plasma from the patient is about 30 μg·h/mL to about 175 μg·h/mL.

In another embodiment, the AUCₛₛ of Compound (1) in plasma from the patient is about 30 μg·h/mL to about 150 μg·h/mL.

In another embodiment, the AUCₛₛ of Compound (1) in plasma from the patient is about 30 μg·h/mL to about 125 μg·h/mL.

In another embodiment, the AUCₛₛ of Compound (1) in plasma from the patient is about 30 μg·h/mL to about 100 μg·h/mL.

In another embodiment, the AUCₛₛ of Compound (1) in plasma from the patient is about 50 μg·h/mL to about 225 μg·h/mL.

In another embodiment, the AUCₛₛ of Compound (1) in plasma from the patient is about 50 μg·h/mL to about 200 μg·h/mL.

In another embodiment, the AUCₛₛ of Compound (1) in plasma from the patient is about 100 μg·h/mL to about 190 μg·h/mL.

In another embodiment, the AUCₛₛ of Compound (1) in plasma from the patient is about 120 μg·h/mL to about 220 μg·h/mL.

In another embodiment, the AUCₛₛ of Compound (1) in plasma from the patient is about 150 μg·h/mL to about 190 μg·h/mL.

In another embodiment, the AUCₛₛ of Compound (1) in plasma from the patient is about 30 μg·h/mL, about 40 μg·h/mL, about 50 μg·h/mL, about 60 μg·h/mL, about 70 μg·h/mL, about 80 μg·h/mL, about 90 μg·h/mL, about 100 μg·h/mL, about 110 μg·h/mL, about 120 μg·h/mL, about 130 μg·h/mL, about 140 μg·h/mL, about 150 μg·h/mL, about 160 μg·h/mL, about 170 μg·h/mL, about 180 μg·h/mL, about 190 μg·h/mL, about 200 μg·h/mL, about 210 μg·h/mL, or about 220 μg·h/mL.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 200 μg·h/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 2306 ng/mL to about 9126 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 3329 ng/mL to about 5716 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 4011 ng/mL to about 7421 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 4864 ng/mL to about 6569 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 5034 ng/mL to about 6569 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 5375 ng/mL to about 6569 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 2306 ng/mL, about 2647 ng/mL, about 2988 ng/mL, about 3329 ng/mL, about 3670 ng/mL, about 4011 ng/mL, about 4352 ng/mL, about 4693 ng/mL, about 4864 ng/mL, about 5034 ng/mL, about 5375 ng/mL, about 5716 ng/mL, about 6569 ng/mL, about 7421 ng/mL, about 8274 ng/mL, or about 9126 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 4352 ng/mL for treating a disease or disorder.

In another embodiment, the C_{min ss} of 5-[[4-[2-[5-(1-hydroxyethyl)pyridin-2-yl]ethoxy]phenyl]methyl]-1,3-thiazolidine-2,4-dione is about 5716 ng/mL for treating a disease or disorder.

In another embodiment, the present disclosure provides methods of treating a disease or disorder, e.g., a non-CNS disease or disorder, or a disease or disorder in a child, in a patient in need thereof, the method comprising administering 5-[[4-[2-[5-acetylpyridin-2-yl]ethoxy]phenyl]methyl]-1,3-thiazolidine-2,4-dione (Compound (1)), or a pharmaceutically acceptable salt thereof, to the patient, wherein 5-[[4-[2-[5-acetylpyridin-2-yl]ethoxy]phenyl]methyl]-1,3-thiazolidine-2,4-dione is metabolized to 5-[[4-[2-[5-(1-hydroxyethyl)pyridin-2-yl]ethoxy]phenyl]methyl]-1,3-thiazolidine-2,4-dione in the patient, and:
(a) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 80 μg·h/mL;
(b) the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 1624 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 80 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 1624 ng/mL; and
the AUCₛₛ of (i), the C_{min ss} of (ii), or the AUCₛₛ and C_{min ss} of (c) is measured after at least five days of administering 5-[[4-[2-[5-acetylpyridin-2-yl]ethoxy]phenyl]methyl]-1,3-thiazolidine-2,4-dione, or a pharmaceutically acceptable salt thereof, to the patient per day.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 40 μg·h/mL for a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 50 μg·h/mL for a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 60 μg·h/mL for a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 70 μg·h/mL for a disease or disorder.

In another embodiment, the AUCₛₛ of 5-[[4-[2-[5-(1-hydroxyethyl)pyridin-2-yl]ethoxy]phenyl]methyl]-1,3-thiazolidine-2,4-dione is about 80 μg·h/mL for a disease or disorder.

In another embodiment, the AUCₛₛ, C_{min ss}, or AUCₛₛ and C_{min ss} is measured after at least seven days.

In another embodiment, the AUCₛₛ, C_{min ss}, or AUCₛₛ and C_{min ss} is measured after at least ten days.

In another embodiment, the AUCₛₛ, C_{min ss}, or AUCₛₛ and C_{min ss} is measured after at least fourteen days.

In another embodiment, the disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(a) administering an amount of Compound (1), or a pharmaceutically acceptable salt thereof, to the patient per day;
(b) obtaining a plasma sample from the patient following 5 days or more of administering according to (a);
(c) determining the C_{min ss} of 5-[[4-[2-[5-(1-hydroxyethyl)pyridin-2-yl]ethoxy]phenyl]methyl]-1,3-thiazolidine-2,4-dione in the plasma sample obtained in (b); and
(d) administering a recalculated amount Compound (1), or a pharmaceutically acceptable salt thereof, to the patient per day as determined according to the Equation 4:

$$D o s {e}_{V 1} = D o s {e}_{p τe - V 1} \times \frac{{C}_{m i {n}_{T A R}}}{{C}_{m i {n}_{V 1}}} , \qquad Equation 4$$

wherein:
Dose_{V1} is the recalculated amount of Compound (1), or a pharmaceutically acceptable salt thereof, administered to the patient per day in (d);
Dose_{pre-V1} is the amount of Compound (1), or a pharmaceutically acceptable salt thereof, administered to the patient in (a);
Cₘᵢₙ_{_{V1}} is the C_{min ss}, in ng/mL, of 5-[[4-[2-[5-(1-hydroxyethyl)pyridin-2-yl]ethoxy]phenyl]methyl]-1,3-thiazolidine-2,4-dione determined in (c) taken 22-26 hours after the last administration; and
Cₘᵢₙ_{_{TAR}} is the targeted concentration in ng/mL of 5-[[4-[2-[5-(1-hydroxyethyl)pyridin-2-yl]ethoxy]phenyl]methyl]-1,3-thiazolidine-2,4-dione, wherein:
(A) Cₘᵢₙ_{_{TAR}} is calculated according to Equation 5A:

Cₘᵢₙ_{_{TAR}}=7700−(88.5×Dose_{pre-V1}) Equation 5A
if the plasma sample in (b) was obtained 18 hours to 19.9 hours after the last administration of Compound (1), or a pharmaceutically acceptable salt thereof, in (a);
(B) Cₘᵢₙ_{_{TAR}} is calculated according to Equation 5B:

Cₘᵢₙ_{_{TAR}}=7440−(103.4×Dose_{pre-V1}) Equation 5B
if the plasma sample in (b) was obtained 20 hours to 21.9 hours after the last administration of Compound (1), or a pharmaceutically acceptable salt thereof, in (a);
(C) Cₘᵢₙ_{_{TAR}} is 5716 if the plasma sample in (b) was obtained 22 hours to 25.9 hours after the last administration of Compound (1), or a pharmaceutically acceptable salt thereof, in (a);
(D) Cₘᵢₙ_{_{TAR}} is calculated according to Equation 5D:

Cₘᵢₙ_{_{TAR}}=6740−(138.6×Dose_{pre-V1}) Equation 5D
if the plasma sample in (b) was obtained 26 hours to 27.9 hours after the last administration of Compound (1), or a pharmaceutically acceptable salt thereof, in (a); or
(E) Cₘᵢₙ_{_{TAR}} is calculated according to Equation 5E:

Cₘᵢₙ_{_{TAR}}=6520−(148.0×Dose_{pre-V1}) Equation 5E
if the plasma sample in (b) was obtained 28 hours to 30 hours after the last administration of Compound (1), or a pharmaceutically acceptable salt thereof, in (a).

In another embodiment in connection with the embodiment immediately above, the disclosure provides a method further comprising:
(i) obtaining a plasma sample from the patient following 5 days or more of administering the recalculated amount Compound (1), or a pharmaceutically acceptable salt thereof, to the patient per day in (d);
(ii) determining the Cₘᵢₙ_{_{calcd}}, in ng/mL, of 5-[[4-[2-[5-(1-hydroxyethyl)pyridin-2-yl]ethoxy]phenyl]methyl]-1,3-thiazolidine-2,4-dione in the plasma sample obtained in (i) according to Equation 6:

$${C}_{m i {n}_{c a l c d}} = \frac{D o s {e}_{V 1} \times {C}_{m i {n}_{V 2}}}{D o s {e}_{l a s t t a k e n}} ; \qquad Equation 6$$

(iii) determining the AUC_{Calcd}, in μg·h/mL, of 5-[[4-[2-[5-(1-hydroxyethyl)pyridin-2-yl]ethoxy]phenyl]methyl]-1,3-thiazolidine-2,4-dione based on the Cₘᵢₙ_{_{calcd}} determined in (ii), wherein:
(A) the AUC_{Calcd} is calculated according to Equation 7A:

$$A U {C}_{C a l c d} = \frac{{C}_{m i {n}_{c a l c d}} + ( 88.5 \times D o s {e}_{l a s t t a k e n} )}{38.5} , \qquad Equation 7 A$$

if the plasma sample in (i) was obtained between 18 hours to 19.9 hours after the last administration of Compound (1), or a pharmaceutically acceptable salt thereof;
(B) the AUC_{Calcd} is calculated according to Equation 7B:

$$A U {C}_{C a l c d} = \frac{{C}_{m i {n}_{c a l c d}} + ( 103.4 \times D o s {e}_{l a s t t a k e n} )}{37.2} , \qquad Equation 7 B$$

if the plasma sample in (i) was obtained 20 hours to 21.9 hours after the last administration of Compound (1), or a pharmaceutically acceptable salt thereof;
(C) the AUC_{Calcd} is calculated according to Equation 7C:

$$A U {C}_{C a l c d} = \frac{{C}_{m i {n}_{c a l c d}} + 1104.1}{34.1} , \qquad Equation 7 C$$

if the plasma sample in (i) was obtained 22 hours to 25.9 hours after the last administration of Compound (1), or a pharmaceutically acceptable salt thereof;
(D) the AUC_{Calcd} is calculated according to Equation 7D:

$$A U {C}_{C a l c d} = \frac{{C}_{m i {n}_{c a l c d}} + ( 138.6 \times D o s {e}_{l a s t t a k e n} )}{33.7} , \qquad Equation 7 D$$

if the plasma sample in (i) was obtained 26 hours to 27.9 hours after the last administration of Compound (1), or a pharmaceutically acceptable salt thereof; or
(E) the AUC_{Calcd} is calculated according to Equation 7E:

$$A U {C}_{C a l c d} = \frac{{C}_{m i {n}_{c a l c d}} + ( 148 \times D o s {e}_{l a s t t a k e n} )}{32.6} , \qquad Equation 7 E$$

if the plasma sample in (i) was obtained 28 hours to 30 hours after the last administration of Compound (1), or a pharmaceutically acceptable salt thereof; and
(iv) administering the same recalculated amount of Compound (1), or a pharmaceutically acceptable salt thereof, to the patient per day as in (i) for 5 days or more if the AUC_{Calcd} is 150 to 240 μg·h/mL and, optionally, repeating (i)-(iii); or
(v) administering an new amount of Compound (1), or a pharmaceutically acceptable salt thereof, to the patient per day in (i) if the AUC_{Calcd} is less than 150 or more than 240 μg·h/mL.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 149 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 241 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 150 μg·h/mL and 240 μg·h/mL.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 159 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 231 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 160 μg·h/mL and 230 μg·h/mL.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for 5 days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 179 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 221 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 180 μg·h/mL and 220 μg·h/mL.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for 5 days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 189 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 211 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 190 μg·h/mL and 210 μg·h/mL.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for 5 days or more; and
(a) increasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is less than 149 μg·h/mL;
(b) decreasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is more than 241 μg·h/mL; or
(c) not changing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is between 150 μg·h/mL and 240 μg·h/mL;
   wherein the increased, decreased, or unchanged dose of Compound (1), or a pharmaceutically acceptable salt thereof, is administered to the patient once per day for at least five days.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for 5 days or more; and
(a) increasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is less than 159 μg·h/mL;
(b) decreasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is more than 231 μg·h/mL; or
(c) not changing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is between 160 μg·h/mL and 230 μg·h/mL;
   wherein the increased, decreased, or unchanged dose of Compound (1), or a pharmaceutically acceptable salt thereof, is administered to the patient once per day for at least five days.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for 5 days or more; and
(a) increasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is less than 179 μg·h/mL;
(b) decreasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is more than 221 μg·h/mL; or
(c) not changing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is between 180 μg·h/mL and 220 μg·h/mL;
   wherein the increased, decreased, or unchanged dose of Compound (1), or a pharmaceutically acceptable salt thereof, is administered to the patient once per day for at least five days.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of Compound (1), or a pharmaceutically acceptable salt thereof, to the patient once per day for 5 days or more; and
(a) increasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is less than 189 μg·h/mL;
(b) decreasing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is more than 211 μg·h/mL; or
(c) not changing the initial dose of Compound (1), or a pharmaceutically acceptable salt thereof, if the plasma concentration of 5-[[4-[2-[5-(1-hydroxyethyl)pyridin-2-yl]ethoxy]phenyl]methyl]-1,3-thiazolidine-2,4-dione in the patient is between 190 μg·h/mL and 210 μg·h/mL;
   wherein the increased, decreased, or unchanged dose of Compound (1), or a pharmaceutically acceptable salt thereof, is administered to the patient once per day for at least five days.

In another embodiment, the present disclosure provides a method, comprising administering 5-[[4-[2-[5-(1-hydroxyethyl)pyridin-2-yl]ethoxy]phenyl]methyl]-1,3-thiazolidine-2,4-dione to a patient in need thereof, wherein:
(a) an initial dose of Compound (1), or a pharmaceutically acceptable salt thereof, to the patient once per day for 5 days or more;
(b) the initial dose of Compound (1), or a pharmaceutically acceptable salt thereof, is increased if the plasma concentration of 5-[[4-[2-[5-(1-hydroxyethyl)pyridin-2-yl]ethoxy]phenyl]methyl]-1,3-thiazolidine-2,4-dione in the patient is less than 149 μg·h/mL;
(c) the initial dose of the Compound (1), or a pharmaceutically acceptable salt thereof, is decreased if the plasma concentration of 5-[[4-[2-[5-(1-hydroxyethyl)pyridin-2-yl]ethoxy]phenyl]methyl]-1,3-thiazolidine-2,4-dione in the patient is more than 241 μg·h/mL; or
(d) the initial dose of Compound (1), or a pharmaceutically acceptable salt thereof, is unchanged if the plasma concentration of 5-[[4-[2-[5-(1-hydroxyethyl)pyridin-2-yl]ethoxy]phenyl]methyl]-1,3-thiazolidine-2,4-dione in the patient is between 150 μg·h/mL and 240 μg·h/mL; and
(e) the increased, decreased, or unchanged dose of Compound (1), or a pharmaceutically acceptable salt thereof, is administered to the patient once per day for 5 days or more.

In another embodiment, the present disclosure provides a method, comprising administering 5-[[4-[2-[5-(1-hydroxyethyl)pyridin-2-yl]ethoxy]phenyl]methyl]-1,3-thiazolidine-2,4-dione to a patient in need thereof, wherein:
(a) an initial dose of Compound (1), or a pharmaceutically acceptable salt thereof, is administered to the patient once per day for 5 days or more;
(b) the initial dose of Compound (1), or a pharmaceutically acceptable salt thereof, is increased if the plasma concentration of 5-[[4-[2-[5-(1-hydroxyethyl)pyridin-2-yl]ethoxy]phenyl]methyl]-1,3-thiazolidine-2,4-dione in the patient is less than 159 μg·h/mL;
(c) the initial dose of Compound (1), or a pharmaceutically acceptable salt thereof, is decreased if the plasma concentration of 5-[[4-[2-[5-(1-hydroxyethyl)pyridin-2-yl]ethoxy]phenyl]methyl]-1,3-thiazolidine-2,4-dione in the patient is more than 231 μg·h/mL; or
(d) the initial dose of Compound (1), or a pharmaceutically acceptable salt thereof, is unchanged if the plasma concentration of 5-[[4-[2-[5-(1-hydroxyethyl)pyridin-2-yl]ethoxy]phenyl]methyl]-1,3-thiazolidine-2,4-dione in the patient is between 160 μg·h/mL and 230 μg·h/mL; and
(e) the increased, decreased, or unchanged dose of Compound (1), or a pharmaceutically acceptable salt thereof, is administered to the patient once per day for 5 days or more.

In another embodiment, the present disclosure provides a method, comprising administering 5-[[4-[2-[5-(1-hydroxyethyl)pyridin-2-yl]ethoxy]phenyl]methyl]-1,3-thiazolidine-2,4-dione to a patient in need thereof, wherein:
(a) an initial dose of Compound (1), or a pharmaceutically acceptable salt thereof, is administered to the patient once per day for 5 days or more;
(b) the initial dose of Compound (1), or a pharmaceutically acceptable salt thereof, is increased if the plasma concentration of 5-[[4-[2-[5-(1-hydroxyethyl)pyridin-2-yl]ethoxy]phenyl]methyl]-1,3-thiazolidine-2,4-dione in the patient is less than 179 μg·h/mL;
(c) the initial dose of Compound (1), or a pharmaceutically acceptable salt thereof, is decreased if the plasma concentration of 5-[[4-[2-[5-(1-hydroxyethyl)pyridin-2-yl]ethoxy]phenyl]methyl]-1,3-thiazolidine-2,4-dione in the patient is more than 221 μg·h/mL; or
(d) the initial dose of Compound (1), or a pharmaceutically acceptable salt thereof, is unchanged if the plasma concentration of 5-[[4-[2-[5-(1-hydroxyethyl)pyridin-2-yl]ethoxy]phenyl]methyl]-1,3-thiazolidine-2,4-dione in the patient is between 180 μg·h/mL and 220 μg·h/mL; and
(e) the increased, decreased, or unchanged dose of Compound (1), or a pharmaceutically acceptable salt thereof, is administered to the patient once per day for 5 days or more.

In another embodiment, the present disclosure provides a method, comprising administering 5-[[4-[2-[5-(1-hydroxyethyl)pyridin-2-yl]ethoxy]phenyl]methyl]-1,3-thiazolidine-2,4-dione to a patient in need thereof, wherein:
(a) an initial dose of Compound (1), or a pharmaceutically acceptable salt thereof, is administered to the patient once per day for 5 days or more;
(b) the initial dose of Compound (1), or a pharmaceutically acceptable salt thereof, is increased if the plasma concentration of 5-[[4-[2-[5-(1-hydroxyethyl)pyridin-2-yl]ethoxy]phenyl]methyl]-1,3-thiazolidine-2,4-dione in the patient is less than 189 μg·h/mL;
(c) the initial dose of Compound (1), or a pharmaceutically acceptable salt thereof, is decreased if the plasma concentration of 5-[[4-[2-[5-(1-hydroxyethyl)pyridin-2-yl]ethoxy]phenyl]methyl]-1,3-thiazolidine-2,4-dione in the patient is more than 211 μg·h/mL; or
(d) the initial dose of Compound (1), or a pharmaceutically acceptable salt thereof, is unchanged if the plasma concentration of 5-[[4-[2-[5-(1-hydroxyethyl)pyridin-2-yl]ethoxy]phenyl]methyl]-1,3-thiazolidine-2,4-dione in the patient is between 190 μg·h/mL and 210 μg·h/mL; and
(e) the increased, decreased, or unchanged dose of Compound (1), or a pharmaceutically acceptable salt thereof, is administered to the patient once per day for 5 days or more.

In another embodiment, the present disclosure provides a method of treating a disease or disorder in a patient in need thereof, the method comprising administering an initial dose of Compound (1), or a pharmaceutically acceptable salt thereof, to the patient once per day for 5 days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 149 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 241 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 150 μg·h/mL and 240 μg·h/mL.

In another embodiment, the present disclosure provides a method of treating a disease or disorder in a patient in need thereof, the method comprising administering an initial dose of Compound (1), or a pharmaceutically acceptable salt thereof, to the patient once per day for 5 days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 159 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 231 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 160 μg·h/mL and 230 μg·h/mL.

In another embodiment, the present disclosure provides a method of treating a disease or disorder in a patient in need thereof, the method comprising administering an initial dose of Compound (1), or a pharmaceutically acceptable salt thereof, to the patient once per day for 5 days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 159 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 231 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 160 μg·h/mL and 230 μg·h/mL.

In another embodiment, the present disclosure provides a method of treating a disease or disorder in a patient in need thereof, the method comprising administering an initial dose of Compound (1), or a pharmaceutically acceptable salt thereof, to the patient once per day for 5 days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 179 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 221 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 180 μg·h/mL and 220 μg·h/mL.

In another embodiment, the present disclosure provides a method of treating a disease or disorder in a patient in need thereof, the method comprising administering an initial dose of Compound (1), or a pharmaceutically acceptable salt thereof, to the patient once per day for 5 days or more; and
(a) administering a higher dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 189 μg·h/mL;
(b) administering a lower dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 211 μg·h/mL; or
(c) administering an unchanged dose of Compound (1), or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 190 μg·h/mL and 210 μg·h/mL.

In some embodiments of the present disclosure, the methods further comprise determining the plasma concentration of 5-[[4-[2-[5-(1-hydroxyethyl)pyridin-2-yl]ethoxy]phenyl]methyl]-1,3-thiazolidine-2,4-dione in the patient, e.g., after administering an initial dose of Compound (1), or a pharmaceutically acceptable salt thereof, to the patient once per day for 5 days or more.

In another embodiment, the present disclosure provides a method of administering a therapeutically effective amount of Compound (1) to a patient in need thereof, the method comprising:
(a) administering an initial dose of 5-[[4-[2-[5-(1-hydroxyethyl)pyridin-2-yl]ethoxy]phenyl]methyl]-1,3-thiazolidine-2,4-dione hydrochloride to the patient once per day for 5 or more days; and
(b) administering a recalculated dose of 5-[[4-[2-[5-(1-hydroxyethyl)pyridin-2-yl]ethoxy]phenyl]methyl]-1,3-thiazolidine-2,4-dione hydrochloride according to:

### (i) Equation 8a:

D_{recal}=Dᵢₙₜᵢₐₗ*(AUC_{Tar}/AUC_0t) Equation 8a

wherein:
D_{recal} is recalculated dose of the 5-[[4-[2-[5-(1-hydroxyethyl)pyridin-2-yl]ethoxy]phenyl]methyl]-1,3-thiazolidine-2,4-dione hydrochloride administered to the patient in milligrams;
Dᵢₙᵢₜᵢₐₗ is the initial dose of the 5-[[4-[2-[5-(1-hydroxyethyl)pyridin-2-yl]ethoxy]phenyl]methyl]-1,3-thiazolidine-2,4-dione hydrochloride administered to the patient in milligrams; AUC_{Tar} is the targeted exposure of Compound (1) in the patient 24 hours after the last administration in (a) in ng·h/ml; and
AUC_0t is the calculated exposure of Compound (1) in the patient 24 hours after the last administration in (a) in ng·h/ml;

### (ii) Equation 8b:

AUC_0t=(28.31+0.472*ΔT)*C+(34410+2234*ΔT)*Dᵢₙᵢₜᵢₐₗ/150 Equation 8b

wherein:
AUC_0t is the calculated exposure of Compound (1) in the patient 24 hours after the last administration in (a) in ng·h/ml;
Dᵢₙᵢₜᵢₐₗ is the initial dose of the 5-[[4-[2-[5-(1-hydroxyethyl)pyridin-2-yl]ethoxy]phenyl]methyl]-1,3-thiazolidine-2,4-dione hydrochloride administered to the patient in milligrams;
C is the plasma concentration of Compound (1) in the patient in ng/ml, wherein the plasma sample is taken from the patient 24±6 hours after the last administration in (a); and
ΔT is the difference between the time the plasma sample is taken from the patient and 24 hours after the last administration in (a) in hours;
wherein the targeted exposure is 50,000 ng·h/mL to 250,000 ng·h/mL.

For example, with respect to ΔT, if the plasma sample was taken 24.5 hours after the last administration the AT would be 0.5 hours. Likewise, if the plasma sample was taken 23 hours after the last administration the AT would be 1 hour.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(a) administering an initial dose of Compound (1), or a pharmaceutically acceptable salt thereof, to the patient once per day for 5 days or more; and
(b) administering a recalculated dose of Compound (1), or a pharmaceutically acceptable salt thereof, according to Equations 8a and 8b, wherein the targeted exposure is 100,000 ng·h/mL to 200,000 ng·h/mL.

In another embodiment, the present disclosure provides a method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(a) administering an initial dose of Compound (1), or a pharmaceutically acceptable salt thereof, to the patient once per day for 5 days or more; and
(b) administering a recalculated dose of Compound (1), or a pharmaceutically acceptable salt thereof, according to Equations 8a and 8b, wherein the targeted exposure is 100,000 ng·h/mL, 120,000 ng·h/mL, 130,000 ng·h/mL, 140,000 ng·h/mL, 150,000 ng·h/mL, 160,000 ng·h/mL, 175,000 ng·h/mL, 180,000 ng·h/mL, 190,000 ng·h/mL, 20 200,000 ng·h/mL.

In another embodiment, the present disclosure provides a method treating a disease or disorder in a patient in need thereof, the method comprising:
(a) administering an initial dose of Compound (1), or a pharmaceutically acceptable salt thereof, to the patient once per day for 5 days or more; and
(b) administering a recalculated dose of Compound (1), or a pharmaceutically acceptable salt thereof, according to Equations 8a and 8b, wherein the targeted exposure is 50,000 ng·h/mL to 250,000 ng·h/mL.

In another embodiment, the present disclosure provides a method treating a disease or disorder in a patient in need thereof, the method comprising:
(a) administering an initial dose of Compound (1), or a pharmaceutically acceptable salt thereof, to the patient once per day for 5 days or more; and
(b) administering a recalculated dose of Compound (1), or a pharmaceutically acceptable salt thereof, according to Equations 8a and 8b, wherein the targeted exposure is 100,000 ng·h/mL to 200,000 ng·h/mL.

In another embodiment, the present disclosure provides a method treating a disease or disorder in a patient in need thereof, the method comprising:
(a) administering an initial dose of Compound (1), or a pharmaceutically acceptable salt thereof, to the patient once per day for 5 days or more; and
(b) administering a recalculated dose of Compound (1), or a pharmaceutically acceptable salt thereof, according to Equations 8a and 8b, wherein the targeted exposure is 100,000 ng·h/mL, 120,000 ng·h/mL, 130,000 ng·h/mL, 140,000 ng·h/mL, 150,000 ng·h/mL, 160,000 ng·h/mL, 175,000 ng·h/mL, 180,000 ng·h/mL, 190,000 ng·h/mL, or 200,000 ng·h/mL.

In another embodiment, 5-[[4-[2-[5-acetylpyridin-2-yl]ethoxy]phenyl]methyl]-1,3-thiazolidine-2,4-dione hydrochloride is administered to the patient in need thereof 5-[[4-[2-[5-acetylpyridin-2-yl]ethoxy]phenyl]methyl]-1,3-thiazolidine-2,4-dione hydrochloride is also referred to herein as “Compound (1) HCl.”

In another embodiment, the Compound (1) HCl is administered to the patient as a suspension comprising about 5-15 mg of Compound (1) HCl per mL.

### Definitions

Various examples and embodiments of the inventive subject matter disclosed here are possible and will be apparent to a person of ordinary skill in the art, given the benefit of this disclosure. In this disclosure reference to “some embodiments,” “certain embodiments,” “certain exemplary embodiments,” “particular embodiments,” and similar phrases each means that those embodiments are non-limiting examples of the inventive subject matter, and there are alternative embodiments which are not excluded.

The articles “a,” “an,” and “the” are used herein to refer to one or to more than one (i.e., to at least one) of the grammatical object of the article. By way of example, “an element” means one element or more than one element.

The word “comprising” is used in a manner consistent with its open-ended meaning, that is, to mean that a given product or process can optionally also have additional features or elements beyond those expressly described. It is understood that wherever embodiments are described with the language “comprising,” otherwise analogous embodiments described in terms of “consisting of” and/or “consisting essentially of” are also contemplated and within the scope of this disclosure.

The term “ameliorate” in the context of this present disclosure is understood as meaning any improvement on the situation of the patient treated.

The term “bid administration” or “BID” means twice daily administration of a therapeutic.

The term “SAD” means a single oral dose administration of a therapeutic.

In the present disclosure, each of the terms “compound of formula (1)”, “ketopioglitazone,” “keto pioglitazone (M-III),” “keto pioglitazone,” and “5-{4-[2-(5-acetylpyridin-2-yl)ethoxy]benzyl}-thiazolidine-2,4-dione” refer to 5-[[4-[2-[5-acetylpyridin-2-yl]ethoxy]phenyl]methyl]-1,3-thiazolidine-2,4-dione, which has the structure depicted above, and any stereoisomer thereof.

In the present disclosure, each of the terms “hydroxypioglitazone,” “hydroxy pioglitazone (M-IV),” “hydroxy pioglitazone,” and “5-[4-[2-(5-(1-hydroxyethyl)-2-pyridinyl)ethoxy]benzyl]-2,4-thiazolidinedione” refer to 5-[[4-[2-[5-(1-hydroxyethyl)pyridin-2-yl]ethoxy]phenyl]methyl]-1,3-thiazolidine-2,4-dione, which has the structure depicted above, and any stereoisomer thereof. The term “MIN-102” refers to the hydrochloride salt of racemic 5-[[4-[2-[5-(1-hydroxyethyl)pyridin-2-yl]ethoxy]phenyl]methyl]-1,3-thiazolidine-2,4-dione.

By an “effective” amount or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The term “treatment” or “to treat” in the context of this specification means to ameliorate or eliminate the disease or one or more symptoms associated with said disease. “Treatment” also encompasses ameliorating or eliminating the physiological sequelae of the disease.

As used herein, the phrase “PK variability” or “pharmacokinetic variability” refer to inter-individual variations of a drugs pharmacokinetic parameters, resulting in different plasma concentration-time profiles after administration of the same dose to different patients.

As used herein, the term “steady-state” refers to the pharmacokinetic situation when the rate of drug administration is equal to the rate of drug elimination.

As used herein, the terms “AUC at steady-state” or “AUCₛₛ” refer to the overall amount of drug in plasma at steady-state.

As used herein, the terms “trough value at steady state” or “C_{min ss}” refer to minimum steady-state plasma drug concentration during a dosage interval.

The term “pharmaceutically acceptable salt” refers to salts prepared from pharmaceutically acceptable inorganic and organic acids.

The term “prevention” or “to prevent” refers to the reduction in the risk of acquiring or developing a given disease or disorder, or the reduction or inhibition of the recurrence or a disease or disorder.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The terms “enantiomerically pure” or “enantiopure” refer to a sample of a chiral substance all of whose molecules (within the limits of detection) have the same chirality sense.

The terms “enantiomerically enriched” or “enantioenriched” refer to a sample of a chiral substance whose enantiomeric ratio is greater than 50:50. Enantiomerically enriched compounds may be enantiomerically pure.

The term “primary mitochondrial disorder” or “PMD” refers to a mitochondrial disease that can occur due to germline mutations in mitochondrial DNA (mtDNA) and/or nuclear DNA (nDNA) genes encoding the electron transport chain (ETC) proteins and therefore the production of adenosine-triphosphate (ATP), the major cellular energy carrier.

The term “secondary mitochondrial disorder” or “SMD” refers to a mitochondrial disease accompanying many pathologic processes not involving oxidative phosphorylation (OXPHOS), including inherited diseases with germline mutations in non-OXPHOS genes. SMD can also be acquired secondary to adverse environmental effects which can cause oxidative stress.

### Diseases and Disorders

The methods and uses of the present disclosure comprise administering 5-[[4-[2-[5-acetylpyridin-2-yl]ethoxy]phenyl]methyl]-1,3-thiazolidine-2,4-dione, or a pharmaceutically acceptable salt thereof, to a patient in need thereof, to treat a variety of diseases or disorders. The methods and uses are based on the discovery that 5-[[4-[2-[5-acetylpyridin-2-yl]ethoxy]phenyl]methyl]-1,3-thiazolidine-2,4-dione metabolizes to 5-[[4-[2-[5-(1-hydroxyethyl)pyridin-2-yl]ethoxy]phenyl]methyl]-1,3-thiazolidine-2,4-dione in the patient.

In one embodiment, the disease or disorder is regulated by peroxisome proliferator-activated receptor gamma (PPAR-γ). PPAR-γ regulates, inter alia, fatty acid storage and glucose metabolism, and has been implicated in the pathology of numerous diseases and disorders.

In another embodiment, the disease or disorder is selected from the group consisting of central nervous system disease or disorder, mitochondrial disease, nonalcoholic steatohepatitis (NASH), nonalcoholic fatty liver disease (NAFLD), chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, and an inflammatory respiratory disease.

In another embodiment, the disease or disorder is a central nervous system disease or disorder. In one embodiment, the central nervous system disorder is selected from the group consisting of a neurodegenerative disease, a cerebrovascular disease, seizure, epilepsy, a viral disease, a neuroinflammatory disease, a brain tumour, a traumatic brain injury, and a rare metabolic disease.

In another embodiment, the disease or disorder is a neurodegenerative disease.

In another embodiment, the disease or disorder is selected from the group consisting of leukodystrophy, amyotrophic lateral sclerosis (ALS), Parkinson's disease, multiple sclerosis, Alzheimer's disease, Huntington's chorea, degenerative ataxia, multiple system atrophy, and a motor neuron disease.

In another embodiment, the disease or disorder is selected from the group consisting of leukodystrophy, amyotrophic lateral sclerosis (ALS), Parkinson's disease, multiple sclerosis, Alzheimer's disease, Huntington's chorea, degenerative ataxia, multiple system atrophy, a motor neuron disease, neuromyelitis optica, NBIA (neurodegeneration and brain iron accumulation disorders), and neuromyopathy.

In another embodiment, the disease or disorder is leukodystrophy, and specifically the disease or disorder is adrenoleukodystrophy (ALD or X-ALD).

In another embodiment, the disease or disorder is a degenerative ataxia, such as Friedreich's ataxia.

In another embodiment, the disease or disorder is a motor neuron disease.

In another embodiment, the disease or disorder is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

In another embodiment, the disease or disorder is cerebrovascular disease selected from the group consisting of global or local ischemia, intracerebral haemorrhage, stroke, and vascular dementia.

In another embodiment, the disease or disorder is a central nervous system disorder selected from the group consisting of a viral disease selected from the group consisting of meningitis, encephalitis, rabies, measles, mumps, poliomyelitis, herpes simplex, and varicella zoster.

In another embodiment, the disease or disorder is a rare metabolic disease selected from the group consisting of organic acidemias, fatty acid disorders and genetic mitochondrial disorders.

In another embodiment, the disease or disorder is nonalcoholic fatty liver disease (NAFLD) or nonalcoholic steatohepatitis (NASH).

In another embodiment, the disease or disorder is a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, or inflammatory respiratory disease

In another embodiment, the disease or disorder is regulated by inhibition of mitochondrial pyruvate carrier (MPC). Mitochondrial diseases are a group of disorders, each of which involves a mitochondrial dysfunction. 5-[[4-[2-[5-(1-hydroxyethyl)pyridin-2-yl]ethoxy]phenyl]methyl]-1,3-thiazolidine-2,4-dione has been found by the inventors to exhibit MPC inhibitory activities and it is thus useful in the treatment of mitochondrial diseases. The activity of 5-[[4-[2-[5-(1-hydroxyethyl)pyridin-2-yl]ethoxy]phenyl]methyl]-1,3-thiazolidine-2,4-dione in treating mitochondrial diseases can be demonstrated in appropriate in vitro or in vivo assays, such as, as described, for example, in Compan et al., Molecular Cell 59:491-501 (2015); Abou-Samra et al., Skeletal Muscle 5:25 (2015); (McGreevy et al., Disease Models & Mechanisms 8:195-213 (2015); Bostick et al., Circulation Research Han. 4/18:121-130 (2008); Bostick et al., Molecular Therapy 17(2):253-261 (2009); Zanou et al., J. Physiol. 593.17:3849-3863 (2010); and Signorini et al., Oxidative Medicine and Cellular Longevity Volume 2014, Article ID 195935, 10 pages (2014).

In another embodiment, the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

In another embodiment, the disease or disorder is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

In another embodiment, the disease or disorder is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy and Bethlem myopathy); DiGeorge syndrome; and neuromuscular disorders (such as limb-girdle muscular dystrophy, inflammatory myopathies, Charcot Marie Tooth (CMT) neuropathy, and drug-induced peripheral neuropathies). In another embodiment, collagen VI muscular dystrophies are selected from Ullrich congenital muscular dystrophy, Bethlem myopathy, oculopharyngeal distal, and Emery-Dreifuss.

In another embodiment, the disease or disorder is a secondary mitochondrial disorder selected from the group consisting of Duchenne muscular dystrophy (DMD) and Becker muscular dystrophy (BMD).

### Pharmaceutical Compositions and Use as a Medicament

Pharmaceutical compositions comprising Compound (1), or a pharmaceutically acceptable salt thereof, and a pharmaceutically acceptable excipient, can be administered by any suitable route of administration. For example, any of oral, intraoral, topical, epicutaneous, subcutaneous, transdermal, intramuscular, parenteral, ocular, rectal, vaginal, inhalation, buccal, sublingual and intranasal delivery routes can be suitable.

The present disclosure also relates to the use of Compound (1), or a pharmaceutically acceptable salt thereof, in the manufacture of a medicament for the treatment or prevention of a disease or disorder in a patient in need thereof.

In one embodiment, pharmaceutical compositions comprising Compound (1), or a pharmaceutically acceptable salt thereof, can be administered orally. Oral forms of pharmaceutical compositions can be solid or liquid. Suitable oral dosage forms include tablets, capsules, pills, granules, suspensions, emulsions, syrups or solutions. The pharmaceutical compositions may be a solid form selected from, e.g., tablets, capsules, pills, or granules. In an embodiment, the oral form is a tablet. In another embodiment, the oral form is an oral solution or suspension. These are advantageous when the patient has difficulty swallowing, for example as a result of the disease or for geriatric and pediatric use. Sublingual preparations are also advantageous.

The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation. Thus, the dose of the active agent will depend on the nature and degree of the condition, the age and condition of the patient, and other factors known to those skilled in the art. A typical daily dose of Compound (1), or a pharmaceutically acceptable salt, for an adult is from about 10 mg to about 500 mg. In one embodiment, the daily dose for an adult is from about 50 mg to about 500 mg. In one embodiment, the daily dose for an adult is from about 100 mg to about 200 mg. Lower daily doses for children and teens can be used, such as for example, from 10 mg to 100 mg.

The pharmaceutical compositions may contain conventional excipients known in the art and may be prepared by conventional methods. A specific compound or mixture of compounds may be selected for a particular route of delivery.

Oral dosage forms may be prepared by combining Compound (1), or a pharmaceutically acceptable salt thereof, in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, stabilizers, and disintegrating agents.

Due to their ease of administration, tablets, caplets, and capsules (such as hard gelatin, HPMC, or starch capsules) represent an embodiment of the solid oral dosage unit forms, in which case solid pharmaceutical excipients are used. If desired, tablets or caplets can be coated by standard aqueous or non-aqueous techniques. These dosage forms can be prepared by any of the methods of pharmacy. In general, pharmaceutical compositions and dosage forms are prepared by uniformly and intimately admixing Compound (1), or a pharmaceutically acceptable salt thereof, with liquid carriers, finely divided solid carriers, or both, and then shaping the product into the desired presentation if necessary.

For example, a tablet can be prepared by compression or molding. Compressed tablets can be prepared by compressing in a suitable machine Compound (1), or a pharmaceutically acceptable salt, in a free-flowing form, such as a powder or granules, optionally mixed with one or more excipients. Molded tablets can be made by molding in a suitable machine a mixture of the powdered compound moistened with an inert liquid diluent.

The pharmaceutical compositions may further comprise one or more other therapeutic agents. Combination treatments may be administered simultaneously, sequentially, or separately, by the same or by different routes, or before, during, and after surgical or intervention procedures.

In one embodiment, the present disclosure provides a pharmaceutical composition comprising Compound (1) HCl as an aqueous suspension.

In another embodiment, the present disclosure provides a pharmaceutical composition comprising Compound (1) HCl, Polysorbate 80, carboxymethylcellulose sodium, and water.

In another embodiment, the present disclosure provides a pharmaceutical composition comprising Compound (1) HCl, colloidal microcrystalline cellulose, and carboxymethylcellulose sodium.

The pharmaceutical compositions of the present disclosure comprising Compound (1) HCl may also, optionally, comprise sweeting agents, e.g., sorbitol powder, saccharin sodium, preservatives, e.g., sodium benzoate, flavorings, pH regulators, e.g., sodium citrate, citric acid monohydrate.

Compound (1), or a pharmaceutically acceptable salt thereof, can be used according to the disclosure when the patient is also administered or in combination with one or more of another therapeutic agent selected from antiinflammatory and analgesic agents, antidiabetics (e.g., metformin), dopamine agonists (e.g. levodopa), MAO-B inhibitors, catechol O-methyltransferase (COMT) inhibitors, anticholinergics, other antiparkinsonians (e.g. amantadine), antiNMDA receptors (e.g. memantine), cholinesterase inhibitors, ACE inhibitors, glutamate antagonist (e.g. riluzole), antioxidants, immunomodulators (e.g. fingolimod, anti CD52, CD25 and CD20 monoclonal antibodies, interferon-β-1a, natalizumab, laquinimod, dimethylfumarate) chemotherapeutics, enzyme replacement therapy agents, substrate reduction therapy agents, corticosteroids, antiproliferatives (e.g. methotrexate), anticonvulsant medications, anticoagulants, antihypertensives and neuroprotectives. The compounds of the disclosure may also be used when the patient is undergoing gene therapy, bone marrow transplantation, deep brain stimulation or radiotherapy.

The one or more therapeutic agents include a sulfonylurea (e.g., glimepiride, glipizide, glyburide), a glinidine (also known as meglitinides), a thiazolidinedione (e.g., pioglitazone, rosiglitazone, lobeglitazone), a dipeptidyl peptidase 4 (DPP4) inhibitor (e.g., sitagliptin, vildagliptin, saxagliptin, linagliptin, gemigliptin, anagliptin, teneligliptin, alogliptin, trelagliptin, dutogliptin, omarigliptin), a sodium/glucose cotransporter 2 (SGLT2) inhibitor (e.g., canagliflozin, dapagliflozin), a glucagon-like peptide-1 (GLP1) receptor agonist (e.g., exenatide, liraglutide, lixisenatide, albiglutide, dulaglutide, taspoglutide, semaglutide), glucagon like peptide-1 (GLP-1), and insulin (e.g., animal insulin preparations extracted from the pancreas of cattle or pigs; human insulin preparations synthesized by genetic engineering using Escherichia coli or yeast; insulin zinc; protamine insulin zinc; insulin fragments or derivatives (e.g., INS-1), and oral insulin preparations.

### PARTICULAR EMBODIMENTS OF THE DISCLOSURE

The disclosure also provides the following particular embodiments relating to methods of treating a disease or disorder in a patient in need thereof.
Embodiment 1. A method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, wherein said method comprises administering Compound (1), or a pharmaceutically acceptable salt thereof, to the patient.
Embodiment 2. The method of Embodiment 1, wherein the method provides an exposure of said 5-[[4-[2-[5-(1-hydroxyethyl)pyridin-2-yl]ethoxy]phenyl]methyl]-1,3-thiazolidine-2,4-dione and Compound (1) in the plasma of the patient at a ratio of about 7:3 (5-[[4-[2-[5-(1-hydroxyethyl)pyridin-2-yl]ethoxy]phenyl]methyl]-1,3-thiazolidine-2,4-dione: Compound (1)).
Embodiment 3. A method of treating or preventing a disease or disorder, comprising administering to a subject in need thereof a dosage form comprising an effective amount of Compound (1), or a pharmaceutically acceptable salt thereof, wherein said disease or disorder is selected from the group consisting of a central nervous system disorder, nonalcoholic fatty liver disease, nonalcoholic steatohepatitis, a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease.
Embodiment 4. The method of Embodiment 3, wherein the central nervous system disorder is selected from the group consisting of a neurodegenerative disease, a cerebrovascular disease, seizure, epilepsy, a viral disease, a neuroinflammatory disease, a brain tumour, a traumatic brain injury, and a rare metabolic disease.
Embodiment 5. The method according to Embodiment 4, wherein the neurodegenerative disease is selected from the group consisting of leukodystrophy, amyotrophic lateral sclerosis (ALS), Parkinson's disease, multiple sclerosis, Alzheimer's disease, Huntington's chorea, degenerative ataxia, multiple system atrophy, and a motor neuron disease.
Embodiment 6. The method of Embodiment 5, wherein the leukodystrophy is adrenoleukodystrophy (ALD or X-ALD).
Embodiment 7. The method of Embodiment 5, wherein the degenerative ataxia is Friedreich's ataxia.
Embodiment 8. The method of Embodiment 5, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).
Embodiment 9. The method of Embodiment 4, wherein the central nervous system disorder is a cerebrovascular disease selected from the group consisting of global or local ischemia, intracerebral haemorrhage, stroke, and vascular dementia.
Embodiment 10. The method of Embodiment 4, the central nervous system disorder is a viral disease selected from the group consisting of meningitis, encephalitis, rabies, measles, mumps, poliomyelitis, herpes simplex, and varicella zoster.
Embodiment 11. The method of Embodiment 4, wherein the rare metabolic disease is selected from the group consisting of organic acidemias, fatty acid disorders and genetic mitochondrial disorders.
Embodiment 12. The method of any one of Embodiments 3-11, wherein said Compound (1) or a pharmaceutically acceptable salt is administered at a daily dose of from about 10 mg to about 500 mg.
Embodiment 13. The method of any one of Embodiments 3-12, wherein said Compound (1) or a pharmaceutically acceptable salt is administered at a daily dose of from about 50 mg to about 500 mg.
Embodiment 14. The method of any one of Embodiments 9-13, wherein a detectable amount of 5-[[4-[2-[5-(1-hydroxyethyl)pyridin-2-yl]ethoxy]phenyl]methyl]-1,3-thiazolidine-2,4-dione is found in the central nervous system (CNS) of the subject after administration.
Embodiment 15. The method of Embodiment 14, wherein said 5-[[4-[2-[5-(1-hydroxyethyl)pyridin-2-yl]ethoxy]phenyl]methyl]-1,3-thiazolidine-2,4-dione is found in the CNS of the subject at an exposure of at least about 100 μg·h/mL after 1 hour after oral administration of a dose of said Compound (1), or a pharmaceutically acceptable salt thereof, wherein said dose is from about 10 mg to about 500 mg.
Embodiment 16. The method of any one of Embodiments 9, 12 or 13, wherein the disease or disorder is nonalcoholic fatty liver disease (NAFLD) or nonalcoholic steatohepatitis (NASH).
Embodiment 17. The method of any one of Embodiments 9, 12, or 13, wherein said disease or disorder is a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, or inflammatory respiratory disease
Embodiment 18. The method of any one of Embodiments 9, 12 or 13, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.
Embodiment 19. The method of Embodiment 18, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).
Embodiment 20. The method of any one of Embodiments 9, 12, or 13, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy and Bethlem myopathy); DiGeorge syndrome; and neuromuscular disorders (such as limb-girdle muscular dystrophy, inflammatory myopathies, Charcot Marie Tooth (CMT) neuropathy, and drug-induced peripheral neuropathies).
Embodiment 21. The method according to any one of Embodiments 9 to 20, further comprising administering another therapeutic agent.
Embodiment 22. The method according to any one of Embodiments 1 to 21, wherein no more than 1% of the total number of hydrogen atoms per mole of said Compound (1) are in the form of the ²H isotope.
Embodiment 23. The method according to any one of Embodiments 1 to 22, wherein said Compound (1), or a pharmaceutically acceptable salt thereof, is administered to the subject in an oral, intraoral, topical, epicutaneous, subcutaneous, transdermal, intramuscular, parenteral, ocular, rectal, vaginal, inhalation, buccal, sublingual, or intranasal dosage form.
Embodiment 24. The method according to Embodiment 23, wherein the dosage form is an oral dosage form.
Embodiment 25. The method according to Embodiment 24, wherein the oral dosage form is solid.
Embodiment 26. The method according to Embodiment 25, wherein the oral solid dosage form is a tablet, a capsule, a pill, or a plurality of granules.
Embodiment 27. The method according to Embodiment 24, wherein the oral dosage form is an oral solution or an oral suspension.
Embodiment 28. A method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(i) administering an amount of Compound (1), or a pharmaceutically acceptable salt thereof, to the patient per day;
(ii) obtaining a plasma sample from the patient after at least 5 days of administering according to (i);
(iii) determining the plasma concentration of 5-[[4-[2-[5-(1-hydroxyethyl)pyridin-2-yl]ethoxy]phenyl]methyl]-1,3-thiazolidine-2,4-dione in the plasma sample obtained in (ii);
(iv) administering a recalculated amount of Compound (1), or a pharmaceutically acceptable salt thereof, in milligrams, to the patient per day as determined as follows:
   according to the Equation 1:

$$n e w a m o u n t in mg = S D \times ( \frac{C M T}{P C} ) , \qquad Equation 1$$

wherein.
SD is the amount of Compound (1), or a pharmaceutically acceptable salt thereof, administered to the patient in (i) in mg;
CMT is the C_{min target} in ng/mL of 5-[[4-[2-[5-(1-hydroxyethyl)pyridin-2-yl]ethoxy]phenyl]methyl]-1,3-thiazolidine-2,4-dione;
C_{min target}=(target AUC ng·h/mL×0.0341±20%)−1104±20%; and
PC is the plasma concentration in ng/mL of 5-[[4-[2-[5-(1-hydroxyethyl)pyridin-2-yl]ethoxy]phenyl]methyl]-1,3-thiazolidine-2,4-dione determined in (iii). In some embodiments, CMT or C_{min target}=(target AUC ng·h/mL×0.0341±10%)−1104±10% in Equation 1. In some embodiments, CMT or C_{min target}=(target AUC ng·h/mL×0.0341±5%)−1104±5% in Equation 1. In some embodiments, CMT or C_{min target}=(target AUC ng·h/mL×0.0341−1104 in Equation 1.
Embodiment 29. A method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(i) administering an amount of Compound (1), or a pharmaceutically acceptable salt thereof, to the patient per day;
(ii) obtaining a plasma sample from the patient after at least 5 days of administering according to (i);
(iii) determining the plasma concentration of 5-[[4-[2-[5-(1-hydroxyethyl)pyridin-2-yl]ethoxy]phenyl]methyl]-1,3-thiazolidine-2,4-dione in the plasma sample obtained in (ii);
(iv) administering a recalculated amount of Compound (1), or a pharmaceutically acceptable salt thereof, in milligrams, to the patient per day as determined according to the Equation 1:

$$n e w a m o u n t in mg = S D \times ( \frac{C M T}{P C} ) , \qquad Equation 1$$

wherein:
SD is the amount of Compound (1), or a pharmaceutically acceptable salt thereof, administered to the patient in (i) in mg;
CMT is the C_{min target} in ng/mL of 5-[[4-[2-[5-(1-hydroxyethyl)pyridin-2-yl]ethoxy]phenyl]methyl]-1,3-thiazolidine-2,4-dione;
C_{min target}=(target AUC ng·h/mL×A)+B, wherein A and B are determined from the linear regression of Cmin and AUC upon oral administration of Compound (1) to humans; and
PC is the plasma concentration in ng/mL of 5-[[4-[2-[5-(1-hydroxyethyl)pyridin-2-yl]ethoxy]phenyl]methyl]-1,3-thiazolidine-2,4-dione determined in (iii).
Embodiment 30. The method of Embodiment 28 or 29, wherein the plasma sample is obtained from the patient after at least 7 days of administering according to (i).
Embodiment 31. The method of Embodiment 30, wherein a plasma sample is obtained from the patient after at least 10 days of administering according to (i).
Embodiment 32. The method of Embodiment 31, wherein a plasma sample is obtained from the patient after at least 14 days of administering according to (i).
Embodiment 33. The method of any one of Embodiments 29-32, wherein the Compound (1), or a pharmaceutically acceptable salt thereof, is administered orally to the patient in (i) and (iv).
Embodiment 34. The method of any one of Embodiments 29-33, wherein 5-[[4-[2-[5-acetylpyridin-2-yl]ethoxy]phenyl]methyl]-1,3-thiazolidine-2,4-dione hydrochloride is administered to the patient per day in (i) and (iv).
Embodiment 35. The method of Embodiment 34, wherein from about 10 to about 500 mg of 5-[[4-[2-[5-acetylpyridin-2-yl]ethoxy]phenyl]methyl]-1,3-thiazolidine-2,4-dione hydrochloride is administered to the patient per day in (i).
Embodiment 36. The method of Embodiment 35, wherein from about 50 to about 500 mg of 5-[[4-[2-[5-acetylpyridin-2-yl]ethoxy]phenyl]methyl]-1,3-thiazolidine-2,4-dione hydrochloride is administered to the patient per day in (i)
Embodiment 37. The method of Embodiment 29, wherein a recalculated amount of 5-[[4-[2-[5-acetylpyridin-2-yl]ethoxy]phenyl]methyl]-1,3-thiazolidine-2,4-dione hydrochloride is administered to the patient per day in (iv).
Embodiment 38. The method of any one of Embodiments 29-33, wherein the 5-[[4-[2-[5-acetylpyridin-2-yl]ethoxy]phenyl]methyl]-1,3-thiazolidine-2,4-dione hydrochloride is administered to the patient in (i) and (iv) as a suspension comprising about 5-15 mg of 5-[[4-[2-[5-acetylpyridin-2-yl]ethoxy]phenyl]methyl]-1,3-thiazolidine-2,4-dione hydrochloride per mL.
Embodiment 39. The method of any one of Embodiments 29-38, wherein the patient has a disease or disorder.
Embodiment 40. A method of treating a disease or disorder in a patient in need thereof, the method comprising administering Compound (1), or a pharmaceutically acceptable salt thereof, to the patient, wherein 5-[[4-[2-[5-acetylpyridin-2-yl]ethoxy]phenyl]methyl]-1,3-thiazolidine-2,4-dione is metabolized to 5-[[4-[2-[5-(1-hydroxyethyl)pyridin-2-yl]ethoxy]phenyl]methyl]-1,3-thiazolidine-2,4-dione in the patient, and:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL;
(b) the minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 100 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; and
the AUCₛₛ of (i), the C_{min ss} of (ii), or the AUCₛₛ and C_{min ss} of (c) is measured after at least five days of orally administering Compound (1), or a pharmaceutically acceptable salt thereof, to the patient per day.
Embodiment 41. The method of Embodiment 40, wherein the AUCₛₛ of 5-[[4-[2-[5-(1-hydroxyethyl)pyridin-2-yl]ethoxy]phenyl]methyl]-1,3-thiazolidine-2,4-dione is about 100 μg·h/mL to about 300 μg·h/mL.
Embodiment 42. The method of Embodiment 41, wherein the AUCₛₛ of 5-[[4-[2-[5-(1-hydroxyethyl)pyridin-2-yl]ethoxy]phenyl]methyl]-1,3-thiazolidine-2,4-dione is about 150 μg·h/mL to about 250 μg·h/mL.
Embodiment 43. The method of Embodiment 42, wherein the AUCₛₛ of 5-[[4-[2-[5-(1-hydroxyethyl)pyridin-2-yl]ethoxy]phenyl]methyl]-1,3-thiazolidine-2,4-dione is about 175 μg·h/mL to about 225 μg·h/mL.
Embodiment 44. The method of Embodiment 43, wherein the AUCₛₛ of 5-[[4-[2-[5-(1-hydroxyethyl)pyridin-2-yl]ethoxy]phenyl]methyl]-1,3-thiazolidine-2,4-dione is about 200 μg·h/mL.
Embodiment 45. The method of any one of Embodiments 40-44, wherein the C_{min ss} of 5-[[4-[2-[5-(1-hydroxyethyl)pyridin-2-yl]ethoxy]phenyl]methyl]-1,3-thiazolidine-2,4-dione is about 2306 to about 9126 ng/mL.
Embodiment 46. The method of any one of Embodiments 40-45, wherein the C_{min ss} of 5-[[4-[2-[5-(1-hydroxyethyl)pyridin-2-yl]ethoxy]phenyl]methyl]-1,3-thiazolidine-2,4-dione is about 5000 to about 6500 ng/mL.
Embodiment 47. The method of any one of Embodiments 40-46, wherein the C_{min ss} of 5-[[4-[2-[5-(1-hydroxyethyl)pyridin-2-yl]ethoxy]phenyl]methyl]-1,3-thiazolidine-2,4-dione is about 5500 to about 6000 ng/mL.
Embodiment 48. The method of any one of Embodiments 40-47, wherein the C_{min ss} of 5-[[4-[2-[5-(1-hydroxyethyl)pyridin-2-yl]ethoxy]phenyl]methyl]-1,3-thiazolidine-2,4-dione is about 5716 ng/mL.
Embodiment 49. The method of any one of Embodiments 40-48, wherein the AUCₛₛ, C_{min ss}, or AUCₛₛ and C_{min ss} is measured after at least seven days.
Embodiment 50. The method of Embodiment 49, wherein the AUCₛₛ, C_{min ss}, or AUCₛₛ and C_{min ss} is measured after at least ten days.
Embodiment 51. The method of Embodiment 50, wherein the AUCₛₛ, C_{min ss}, or AUCₛₛ and C_{min ss} is measured after at least fourteen days.
Embodiment 52. The method of any one of Embodiments 40-51, wherein 5-[[4-[2-[5-acetylpyridin-2-yl]ethoxy]phenyl]methyl]-1,3-thiazolidine-2,4-dione hydrochloride is administered to the patient in need thereof.
Embodiment 53. The method of Embodiment 52, wherein the 5-[[4-[2-[5-acetylpyridin-2-yl]ethoxy]phenyl]methyl]-1,3-thiazolidine-2,4-dione hydrochloride is administered to the patient as a suspension comprising about 5-15 mg of 5-[[4-[2-[5-acetylpyridin-2-yl]ethoxy]phenyl]methyl]-1,3-thiazolidine-2,4-dione hydrochloride per mL.
Embodiment 54. The method of any one of Embodiments 39-53, wherein the disease or disorder is selected from the group consisting of central nervous system disease or disorder, mitochondrial disease, nonalcoholic steatohepatitis (NASH), nonalcoholic fatty liver disease (NAFLD), chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, and an inflammatory respiratory disease.
Embodiment 55. The method of Embodiment 54, wherein the central nervous system disorder is selected from the group consisting of a neurodegenerative disease, a cerebrovascular disease, seizure, epilepsy, a viral disease, a neuroinflammatory disease, a brain tumour, a traumatic brain injury, and a rare metabolic disease.
Embodiment 56. The method according to Embodiment 55, wherein the neurodegenerative disease is selected from the group consisting of leukodystrophy, amyotrophic lateral sclerosis (ALS), Parkinson's disease, multiple sclerosis, Alzheimer's disease, Huntington's chorea, degenerative ataxia, multiple system atrophy, and a motor neuron disease.
Embodiment 57. The method of Embodiment 56, wherein the leukodystrophy is adrenoleukodystrophy (ALD or X-ALD).
Embodiment 58. The method of Embodiment 56, wherein the degenerative ataxia is Friedreich's ataxia.
Embodiment 59. The method of Embodiment 56, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).
Embodiment 60. The method of Embodiment 55 wherein the central nervous system disorder is a cerebrovascular disease selected from the group consisting of global or local ischemia, intracerebral haemorrhage, stroke, and vascular dementia.
Embodiment 61. The method of Embodiment 55, the central nervous system disorder is a viral disease selected from the group consisting of meningitis, encephalitis, rabies, measles, mumps, poliomyelitis, herpes simplex, and varicella zoster.
Embodiment 62. The method of Embodiment 55, wherein the rare metabolic disease is selected from the group consisting of organic acidemias, fatty acid disorders and genetic mitochondrial disorders.
Embodiment 63. The method of Embodiment 54, wherein the disease or disorder is nonalcoholic fatty liver disease (NAFLD) or nonalcoholic steatohepatitis (NASH).
Embodiment 64. The method of Embodiment 54, wherein said disease or disorder is a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, or inflammatory respiratory disease
Embodiment 65. The method of Embodiment 54, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.
Embodiment 66. The method of Embodiment 65, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).
Embodiment 67. The method of Embodiment 54, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy and Bethlem myopathy); DiGeorge syndrome; and neuromuscular disorders (such as limb-girdle muscular dystrophy, inflammatory myopathies, Charcot Marie Tooth (CMT) neuropathy, and drug-induced peripheral neuropathies).
Embodiment 68. An oral dosage form, comprising an effective amount of Compound (1), or a pharmaceutically acceptable salt thereof, wherein the effective amount provides the following:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient of about 34 μg·h/mL to about 300 μg·h/mL;
(b) the minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient of about 55 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient of about 34 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient of about 55 to about 9126 ng/mL; and
the AUCₛₛ of (i), the C_{min ss} of (ii), or the AUCₛₛ and C_{min ss} of (c) is measured after at least five days of orally administering Compound (1), or a pharmaceutically acceptable salt thereof, to the patient per day.
Embodiment 69. The oral dosage form of Embodiment 68, comprising from about 10 to about 500 mg of Compound (1), or a pharmaceutically acceptable salt thereof.
Embodiment 70. The oral dosage form of Embodiment 68 or 69, comprising from about 50 to about 500 mg of Compound (1), or a pharmaceutically acceptable salt thereof.
Embodiment 71. The oral dosage form of any one of Embodiments 68-70, wherein the oral dosage form is solid.
Embodiment 72. The oral dosage form of Embodiment 71, wherein the oral solid dosage form is a tablet, a capsule, a pill, or a plurality of granules.
Embodiment 73. The oral dosage form of any one of Embodiments 68-70, wherein the oral dosage form is an oral solution or an oral suspension.

The disclosure also provides the following particular “Use Embodiments” relating to Compound (1), or a pharmaceutically acceptable salt thereof, for use in treating a disease or disorder, or for administering a therapeutically effective amount of this drug and/or 5-[[4-[2-[5-(1-hydroxyethyl)pyridin-2-yl]ethoxy]phenyl]methyl]-1,3-thiazolidine-2,4-dione to a patient.
Use Embodiment 1. Compound (1), or a pharmaceutically acceptable salt thereof, for use in administering a therapeutically effective amount of 5-[[4-[2-[5-(1-hydroxyethyl)pyridin-2-yl]ethoxy]phenyl]methyl]-1,3-thiazolidine-2,4-dione to a patient.
Use Embodiment 2. The Compound (1), or a pharmaceutically acceptable salt thereof, for use in Use Embodiment 1, wherein the exposure of said 5-[[4-[2-[5-(1-hydroxyethyl)pyridin-2-yl]ethoxy]phenyl]methyl]-1,3-thiazolidine-2,4-dione and Compound (1) in the plasma of the patient is at a ratio of about 7:3 (5-[[4-[2-[5-(1-hydroxyethyl)pyridin-2-yl]ethoxy]phenyl]methyl]-1,3-thiazolidine-2,4-dione: Compound (1)).
Use Embodiment 3. Compound (1), or a pharmaceutically acceptable salt thereof, for use in treating or preventing a disease or disorder in a subject, wherein said disease or disorder is selected from the group consisting of a central nervous system disorder, nonalcoholic fatty liver disease, nonalcoholic steatohepatitis, a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease.
Use Embodiment 4. The Compound (1), or a pharmaceutically acceptable salt thereof, for use of Use Embodiment 3, wherein the central nervous system disorder is selected from the group consisting of a neurodegenerative disease, a cerebrovascular disease, seizure, epilepsy, a viral disease, a neuroinflammatory disease, a brain tumour, a traumatic brain injury, and a rare metabolic disease.
Use Embodiment 5. The Compound (1), or a pharmaceutically acceptable salt thereof, for use of Use Embodiment 4, wherein the neurodegenerative disease is selected from the group consisting of leukodystrophy, amyotrophic lateral sclerosis (ALS), Parkinson's disease, multiple sclerosis, Alzheimer's disease, Huntington's chorea, degenerative ataxia, multiple system atrophy, and a motor neuron disease.
Use Embodiment 6. The Compound (1), or a pharmaceutically acceptable salt thereof, for use of Use Embodiment 5, wherein the leukodystrophy is adrenoleukodystrophy (ALD or X-ALD).
Use Embodiment 7. The Compound (1), or a pharmaceutically acceptable salt thereof, for use of Use Embodiment 5, wherein the degenerative ataxia is Friedreich's ataxia.
Use Embodiment 8. The Compound (1), or a pharmaceutically acceptable salt thereof, for use of Use Embodiment 5, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).
Use Embodiment 9. The Compound (1), or a pharmaceutically acceptable salt thereof, for use of Use Embodiment 4, wherein the central nervous system disorder is a cerebrovascular disease selected from the group consisting of global or local ischemia, intracerebral haemorrhage, stroke, and vascular dementia.
Use Embodiment 10. The Compound (1), or a pharmaceutically acceptable salt thereof, for use of Use Embodiment 4, the central nervous system disorder is a viral disease selected from the group consisting of meningitis, encephalitis, rabies, measles, mumps, poliomyelitis, herpes simplex, and varicella zoster.
Use Embodiment 11. The Compound (1), or a pharmaceutically acceptable salt thereof, for use of Use Embodiment 4, wherein the rare metabolic disease is selected from the group consisting of organic acidemias, fatty acid disorders and genetic mitochondrial disorders.
Use Embodiment 12. The Compound (1), or a pharmaceutically acceptable salt thereof, for use of any one of Use Embodiments 3-11, wherein said Compound (1), or a pharmaceutically acceptable salt, is administered at a daily dose of from about 10 mg to about 500 mg.
Use Embodiment 13. The Compound (1), or a pharmaceutically acceptable salt thereof, for use of any one of Use Embodiments 3-12, wherein said Compound (1) or a pharmaceutically acceptable salt is administered at a daily dose of from about 50 mg to about 500 mg.
Use Embodiment 14. The Compound (1), or a pharmaceutically acceptable salt thereof, for use of any one of Use Embodiments 9-13, wherein a detectable amount of 5-[[4-[2-[5-(1-hydroxyethyl)pyridin-2-yl]ethoxy]phenyl]methyl]-1,3-thiazolidine-2,4-dione is found in the central nervous system (CNS) of the subject after administration.
Use Embodiment 15. The Compound (1), or a pharmaceutically acceptable salt thereof, for use of Use Embodiment 14, wherein said 5-[[4-[2-[5-(1-hydroxyethyl)pyridin-2-yl]ethoxy]phenyl]methyl]-1,3-thiazolidine-2,4-dione is found in the CNS of the subject at an exposure of at least about 100 μg·h/mL after 1 hour after oral administration of a dose of said Compound (1), or a pharmaceutically acceptable salt thereof, wherein said dose is from about 10 mg to about 500 mg.
Use Embodiment 16. The Compound (1), or a pharmaceutically acceptable salt thereof, for use of any one of Use Embodiments 9, 12 or 13, wherein the disease or disorder is nonalcoholic fatty liver disease (NAFLD) or nonalcoholic steatohepatitis (NASH).
Use Embodiment 17. The Compound (1), or a pharmaceutically acceptable salt thereof, for use of any one of Use Embodiments 9, 12, or 13, wherein said disease or disorder is a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, or inflammatory respiratory disease
Use Embodiment 18. The Compound (1), or a pharmaceutically acceptable salt thereof, for use of any one of Use Embodiments 9, 12 or 13, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.
Use Embodiment 19. The Compound (1), or a pharmaceutically acceptable salt thereof, for use of Use Embodiment 18, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).
Use Embodiment 20. The Compound (1), or a pharmaceutically acceptable salt thereof, for use of any one of Use Embodiments 9, 12, or 13, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy and Bethlem myopathy); DiGeorge syndrome; and neuromuscular disorders (such as limb-girdle muscular dystrophy, inflammatory myopathies, Charcot Marie Tooth (CMT) neuropathy, and drug-induced peripheral neuropathies).
Use Embodiment 21. The Compound (1), or a pharmaceutically acceptable salt thereof, for use any one of Use Embodiments 9 to 20, further comprising administering another therapeutic agent.
Use Embodiment 22. The Compound (1), or a pharmaceutically acceptable salt thereof, for use of any one of Use Embodiments 1 to 21, wherein no more than 1% of the total number of hydrogen atoms per mole of said Compound (1) are in the form of the ²H isotope.
Use Embodiment 23. The Compound (1), or a pharmaceutically acceptable salt thereof, for use of any one of Use Embodiments 1 to 22, wherein said Compound (1), or a pharmaceutically acceptable salt thereof, is administered to the subject in an oral, intraoral, topical, epicutaneous, subcutaneous, transdermal, intramuscular, parenteral, ocular, rectal, vaginal, inhalation, buccal, sublingual, or intranasal dosage form.
Use Embodiment 24. The Compound (1), or a pharmaceutically acceptable salt thereof, for use of Use Embodiment 23, wherein the dosage form is an oral dosage form.
Use Embodiment 25. The Compound (1), or a pharmaceutically acceptable salt thereof, for use of Use Embodiment 24, wherein the oral dosage form is solid.
Use Embodiment 26. The Compound (1), or a pharmaceutically acceptable salt thereof, for use of Use Embodiment 25, wherein the oral solid dosage form is a tablet, a capsule, a pill, or a plurality of granules.
Use Embodiment 27. The Compound (1), or a pharmaceutically acceptable salt thereof, for use of Use Embodiment 24, wherein the oral dosage form is an oral solution or an oral suspension.
Use Embodiment 28. Compound (1), or a pharmaceutically acceptable salt thereof, for use in administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(i) administering an amount of Compound (1), or a pharmaceutically acceptable salt thereof, to the patient per day;
(ii) obtaining a plasma sample from the patient after at least 5 days of administering according to (i);
(iii) determining the plasma concentration of 5-[[4-[2-[5-(1-hydroxyethyl)pyridin-2-yl]ethoxy]phenyl]methyl]-1,3-thiazolidine-2,4-dione in the plasma sample obtained in (ii);
(iv) administering a recalculated amount of Compound (1), or a pharmaceutically acceptable salt thereof, in milligrams, to the patient per day as determined as according to the Equation 1:

$$n e w a m o u n t in mg = S D \times ( \frac{C M T}{P C} ) , \qquad Equation 1$$

wherein:
SD is the amount of Compound (1), or a pharmaceutically acceptable salt thereof, administered to the patient in (i) in mg;
CMT is the C_{min target} in ng/mL of 5-[[4-[2-[5-(1-hydroxyethyl)pyridin-2-yl]ethoxy]phenyl]methyl]-1,3-thiazolidine-2,4-dione
C_{min target}=(target AUC ng·h/mL×0.0341±20%)−1104±20%; and
PC is the plasma concentration in ng/mL of 5-[[4-[2-[5-(1-hydroxyethyl)pyridin-2-yl]ethoxy]phenyl]methyl]-1,3-thiazolidine-2,4-dione determined in (iii) In some embodiments, CMT or C_{min target}=(target AUC ng·h/mL×0.0341±10%)−1104±10% in Equation 1. In some embodiments, CMT or C_{min target}=(target AUC ng·h/mL×0.0341±5%)−1104±5% in Equation 1. In some embodiments, CMT or C_{min target}=(target AUC ng·h/mL×0.0341−1104 in Equation 1.
Use Embodiment 29. The Compound (1), or a pharmaceutically acceptable salt thereof, for use of Use Embodiment 28, wherein the plasma sample is obtained from the patient after at least 7 days of administering according to (i).
Use Embodiment 30. The Compound (1), or a pharmaceutically acceptable salt thereof, for use of Use Embodiment 29, wherein a plasma sample is obtained from the patient after at least 10 days of administering according to (i).
Use Embodiment 31. The Compound (1), or a pharmaceutically acceptable salt thereof, for use of Use Embodiment 30, wherein a plasma sample is obtained from the patient after at least 14 days of administering according to (i).
Use Embodiment 32. The Compound (1), or a pharmaceutically acceptable salt thereof, for use of any one of Use Embodiments 28-31, wherein the Compound (1), or a pharmaceutically acceptable salt thereof, is administered orally to the patient in (i) and (iv).
Use Embodiment 33. The Compound (1), or a pharmaceutically acceptable salt thereof, for use of any one of Use Embodiments 28-32, wherein 5-[[4-[2-[5-acetylpyridin-2-yl]ethoxy]phenyl]methyl]-1,3-thiazolidine-2,4-dione hydrochloride is administered to the patient per day in (i) and (iv).
Use Embodiment 34. The Compound (1), or a pharmaceutically acceptable salt thereof, for use of Use Embodiment 33, wherein from about 10 to about 500 mg of 5-[[4-[2-[5-acetylpyridin-2-yl]ethoxy]phenyl]methyl]-1,3-thiazolidine-2,4-dione hydrochloride is administered to the patient per day in (i) and the target AUC is about 200 μg·h/mL.
Use Embodiment 35. The Compound (1), or a pharmaceutically acceptable salt thereof, for use of Use Embodiment 34, wherein from about 50 to about 500 mg of 5-[[4-[2-[5-acetylpyridin-2-yl]ethoxy]phenyl]methyl]-1,3-thiazolidine-2,4-dione hydrochloride is administered to the patient per day in (i) and the target AUC is about 200 μg·h/mL.
Use Embodiment 36. The Compound (1), or a pharmaceutically acceptable salt thereof, for use of Use Embodiments 28, or 33-35, wherein a recalculated amount of 5-[[4-[2-[5-acetylpyridin-2-yl]ethoxy]phenyl]methyl]-1,3-thiazolidine-2,4-dione hydrochloride is administered to the patient per day in (iv).
Use Embodiment 37. The Compound (1), or a pharmaceutically acceptable salt thereof, for use of any one of Use Embodiments 28-32, wherein the 5-[[4-[2-[5-acetylpyridin-2-yl]ethoxy]phenyl]methyl]-1,3-thiazolidine-2,4-dione hydrochloride is administered to the patient in (i) and (iv) as a suspension comprising about 15 mg of 5-[[4-[2-[5-acetylpyridin-2-yl]ethoxy]phenyl]methyl]-1,3-thiazolidine-2,4-dione hydrochloride per mL.
Use Embodiment 38. The Compound (1), or a pharmaceutically acceptable salt thereof, for use of any one of Use Embodiments 28-37, wherein the patient has a disease or disorder.
Use Embodiment 39. Compound (1), or a pharmaceutically acceptable salt thereof, for use in treating a disease or disorder in a patient, wherein Compound (1) is metabolized to 5-[[4-[2-[5-(1-hydroxyethyl)pyridin-2-yl]ethoxy]phenyl]methyl]-1,3-thiazolidine-2,4-dione in the patient, and:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL;
(b) the minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; and
the AUCₛₛ of (i), the C_{min ss} of (ii), or the AUCₛₛ and C_{min ss} of (c) is measured after at least five days of orally administering Compound (1), or a pharmaceutically acceptable salt thereof, to the patient per day.
Use Embodiment 40. The Compound (1), or a pharmaceutically acceptable salt thereof, for use of Use Embodiment 39, wherein the AUCₛₛ of 5-[[4-[2-[5-(1-hydroxyethyl)pyridin-2-yl]ethoxy]phenyl]methyl]-1,3-thiazolidine-2,4-dione is about 100 μg·h/mL to about 300 μg·h/mL.
Use Embodiment 41. The Compound (1), or a pharmaceutically acceptable salt thereof, for use of Use Embodiment 40, wherein the AUCₛₛ of 5-[[4-[2-[5-(1-hydroxyethyl)pyridin-2-yl]ethoxy]phenyl]methyl]-1,3-thiazolidine-2,4-dione is about 150 μg·h/mL to about 250 μg·h/mL.
Use Embodiment 42. The Compound (1), or a pharmaceutically acceptable salt thereof, for use of Use Embodiment 41, wherein the AUCₛₛ of 5-[[4-[2-[5-(1-hydroxyethyl)pyridin-2-yl]ethoxy]phenyl]methyl]-1,3-thiazolidine-2,4-dione is about 175 μg·h/mL to about 225 μg·h/mL.
Use Embodiment 43. The Compound (1), or a pharmaceutically acceptable salt thereof, for use of Use Embodiment 42, wherein the AUCₛₛ of 5-[[4-[2-[5-(1-hydroxyethyl)pyridin-2-yl]ethoxy]phenyl]methyl]-1,3-thiazolidine-2,4-dione is about 200 μg·h/mL.
Use Embodiment 44. The Compound (1), or a pharmaceutically acceptable salt thereof, for use of any one of Use Embodiments 39-43, wherein the C_{min ss} of 5-[[4-[2-[5-(1-hydroxyethyl)pyridin-2-yl]ethoxy]phenyl]methyl]-1,3-thiazolidine-2,4-dione is about 2306 to about 9126 ng/mL.
Use Embodiment 45. The Compound (1), or a pharmaceutically acceptable salt thereof, for use of any one of Use Embodiments 39-44, wherein the C_{min ss} of 5-[[4-[2-[5-(1-hydroxyethyl)pyridin-2-yl]ethoxy]phenyl]methyl]-1,3-thiazolidine-2,4-dione is about 5000 to about 6500 ng/mL.
Use Embodiment 46. The Compound (1), or a pharmaceutically acceptable salt thereof, for use of any one of Use Embodiments 39-45, wherein the C_{min ss} of 5-[[4-[2-[5-(1-hydroxyethyl)pyridin-2-yl]ethoxy]phenyl]methyl]-1,3-thiazolidine-2,4-dione is about 5500 to about 6000 ng/mL.
Use Embodiment 47. The Compound (1), or a pharmaceutically acceptable salt thereof, for use of any one of Use Embodiments 39-46, wherein the C_{min ss} of 5-[[4-[2-[5-(1-hydroxyethyl)pyridin-2-yl]ethoxy]phenyl]methyl]-1,3-thiazolidine-2,4-dione is about 5716 ng/mL.
Use Embodiment 48. The Compound (1), or a pharmaceutically acceptable salt thereof, for use of any one of Use Embodiments 39-47, wherein the AUCₛₛ, C_{min ss}, or AUCₛₛ and C_{min ss} is measured after at least seven days.
Use Embodiment 49. The Compound (1), or a pharmaceutically acceptable salt thereof, for use of Use Embodiment 48, wherein the AUCₛₛ, C_{min ss}, or AUCₛₛ and C_{min ss} is measured after at least ten days.
Use Embodiment 50. The Compound (1), or a pharmaceutically acceptable salt thereof, for use of Use Embodiment 49, wherein the AUCₛₛ, C_{min ss}, or AUCₛₛ and C_{min ss} is measured after at least fourteen days.
Use Embodiment 51. The Compound (1), or a pharmaceutically acceptable salt thereof, for use of any one of Use Embodiments 39-50, wherein 5-[[4-[2-[5-acetylpyridin-2-yl]ethoxy]phenyl]methyl]-1,3-thiazolidine-2,4-dione hydrochloride is administered to the patient in need thereof.
Use Embodiment 52. The Compound (1), or a pharmaceutically acceptable salt thereof, for use of Use Embodiment 51, wherein the 5-[[4-[2-[5-acetylpyridin-2-yl]ethoxy]phenyl]methyl]-1,3-thiazolidine-2,4-dione hydrochloride is administered to the patient as a suspension comprising about 15 mg of 5-[[4-[2-[5-acetylpyridin-2-yl]ethoxy]phenyl]methyl]-1,3-thiazolidine-2,4-dione hydrochloride per mL.
Use Embodiment 53. The Compound (1), or a pharmaceutically acceptable salt thereof, for use of any one of Use Embodiments 38-52, wherein the disease or disorder is selected from the group consisting of central nervous system disease or disorder, mitochondrial disease, nonalcoholic steatohepatitis (NASH), nonalcoholic fatty liver disease (NAFLD), chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, and an inflammatory respiratory disease.
Use Embodiment 54. The Compound (1), or a pharmaceutically acceptable salt thereof, for use of Use Embodiment 53, wherein the central nervous system disorder is selected from the group consisting of a neurodegenerative disease, a cerebrovascular disease, seizure, epilepsy, a viral disease, a neuroinflammatory disease, a brain tumour, a traumatic brain injury, and a rare metabolic disease.
Use Embodiment 55. The Compound (1), or a pharmaceutically acceptable salt thereof, for use according to Use Embodiment 54, wherein the neurodegenerative disease is selected from the group consisting of leukodystrophy, amyotrophic lateral sclerosis (ALS), Parkinson's disease, multiple sclerosis, Alzheimer's disease, Huntington's chorea, degenerative ataxia, multiple system atrophy, and a motor neuron disease.
Use Embodiment 56. The Compound (1), or a pharmaceutically acceptable salt thereof, for use of Use Embodiment 55, wherein the leukodystrophy is adrenoleukodystrophy (ALD or X-ALD).
Use Embodiment 57. The Compound (1), or a pharmaceutically acceptable salt thereof, for use of Use Embodiment 55, wherein the degenerative ataxia is Friedreich's ataxia.
Use Embodiment 58. The Compound (1), or a pharmaceutically acceptable salt thereof, for use of Use Embodiment 55, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).
Use Embodiment 59. The Compound (1), or a pharmaceutically acceptable salt thereof, for use of Use Embodiment 54, wherein the central nervous system disorder is a cerebrovascular disease selected from the group consisting of global or local ischemia, intracerebral haemorrhage, stroke, and vascular dementia.
Use Embodiment 60. The Compound (1), or a pharmaceutically acceptable salt thereof, for use of Use Embodiment 54, the central nervous system disorder is a viral disease selected from the group consisting of meningitis, encephalitis, rabies, measles, mumps, poliomyelitis, herpes simplex, and varicella zoster.
Use Embodiment 61. The Compound (1), or a pharmaceutically acceptable salt thereof, for use of Use Embodiment 54, wherein the rare metabolic disease is selected from the group consisting of organic acidemias, fatty acid disorders and genetic mitochondrial disorders.
Use Embodiment 62. The Compound (1), or a pharmaceutically acceptable salt thereof, for use of Use Embodiment 53, wherein the disease or disorder is nonalcoholic fatty liver disease (NAFLD) or nonalcoholic steatohepatitis (NASH).
Use Embodiment 63. The Compound (1), or a pharmaceutically acceptable salt thereof, for use of Use Embodiment 53, wherein said disease or disorder is a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, or inflammatory respiratory disease
Use Embodiment 64. The Compound (1), or a pharmaceutically acceptable salt thereof, for use of Use Embodiment 53, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.
Use Embodiment 65. The Compound (1), or a pharmaceutically acceptable salt thereof, for use of Use Embodiment 64, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).
Use Embodiment 66. The Compound (1), or a pharmaceutically acceptable salt thereof, for use of Use Embodiment 53, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy and Bethlem myopathy); DiGeorge syndrome; and neuromuscular disorders (such as limb-girdle muscular dystrophy, inflammatory myopathies, Charcot Marie Tooth (CMT) neuropathy, and drug-induced peripheral neuropathies).
Use Embodiment 67. An oral dosage form, comprising an effective amount of Compound (1), or a pharmaceutically acceptable salt thereof, wherein the effective amount provides the following:
(a) the steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient of about 34 μg·h/mL to about 300 μg·h/mL;
(b) the minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient of about 55 to about 9126 ng/mL; or
(c) the AUCₛₛ of 5-[[4-[2-[5-(1-hydroxyethyl)pyridin-2-yl]ethoxy]phenyl]methyl]-1,3-thiazolidine-2,4-dione in plasma from the patient of about 34 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient of about 55 to about 9126 ng/mL; and
the AUCₛₛ of (i), the C_{min ss} of (ii), or the AUCₛₛ and C_{min ss} of (c) is measured after at least five days of orally administering Compound (1), or a pharmaceutically acceptable salt thereof, to the patient per day.
Use Embodiment 68. The oral dosage form of Use Embodiment 67, comprising from about 10 to about 500 mg of Compound (1), or a pharmaceutically acceptable salt thereof.
Use Embodiment 69. The oral dosage form of Use Embodiment 67 or 68, comprising from about 50 to about 500 mg of Compound (1), or a pharmaceutically acceptable salt thereof.
Use Embodiment 70. The oral dosage form of any one of Use Embodiments 67-69, wherein the oral dosage form is solid.
Use Embodiment 71. The oral dosage form of Use Embodiment 70, wherein the oral solid dosage form is a tablet, a capsule, a pill, or a plurality of granules.
Use Embodiment 72. The oral dosage form of any one of Use Embodiments 67-69, wherein the oral dosage form is an oral solution or an oral suspension.

### EXAMPLES

The methods of treatment or prevention and uses described herein are now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### Example 1

### The metabolization of 5-[[4-[2-[5-acetylpyridin-2-yl]ethoxy]phenyl]methyl]-1,3-thiazolidine-2,4-dione (M-III) to 5-[[4-[2-[5-(1-hydroxyethyl)pyridin-2-yl]ethoxy]phenyl]methyl]-1,3-thiazolidine-2,4-dione (M-IV)

To the plasma pharmacokinetics of 5-[[4-[2-[5-acetylpyridin-2-yl]ethoxy]phenyl]methyl]-1,3-thiazolidine-2,4-dione (M-III) and 5-[[4-[2-[5-(1-hydroxyethyl)pyridin-2-yl]ethoxy]phenyl]methyl]-1,3-thiazolidine-2,4-dione (M-IV) were determined in male C57BL/6 mice following a single oral (50 mg/kg) dose administration of M-III.

Nine animals were administered orally with suspension formulation of M-III in 0.1% Tween 80 and 99.9% NaCMC in water (0.5% w/v) at 50 mg/kg dose. The dosing volume administered was 10 mL/kg. The blood samples were collected from set of three mice at each time point in labeled micro centrifuge tube containing K₂EDTA solution as anticoagulant at Pre-dose, 0.25, 0.5, 1, 2, 4, 8, 12 and 24 hr. Plasma samples were separated by centrifugation of whole blood and stored below −70±10° C. until bioanalysis. All samples were processed for analysis by protein precipitation using acetonitrile and analyzed with fit-for-purpose LC-MS/MS method (LLOQ=2.46 ng/mL for M-III and 2.49 ng/mL for M-IV). Pharmacokinetic parameters were calculated using the non-compartmental analysis tool of Phoenix WinNonlin® (Version 6.3).

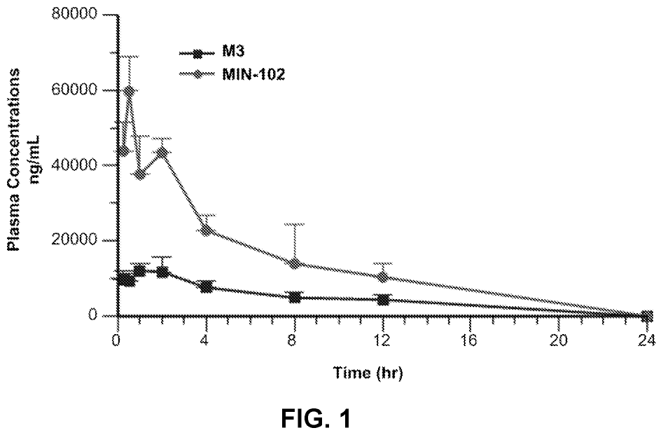

Mean plasma concentration-time profiles of M-III and M-IV following a single oral dose administration of M-III to male C57BL/6 mice (Dose: 50 mg/kg) are shown in in FIG. 1. As can be seen in FIG. 1, M-III is metabolized to M-IV after oral administration. In terms of exposure (AUC), M-IV represents about 75% of the total exposure and M-III represents about 25% of the total exposure.

### Example 2

The plasma pharmacokinetics of 5-[[4-[2-[5-acetylpyridin-2-yl]ethoxy]phenyl]methyl]-1,3-thiazolidine-2,4-dione (M-III) and 5-[[4-[2-[5-(1-hydroxyethyl)pyridin-2-yl]ethoxy]phenyl]methyl]-1,3-thiazolidine-2,4-dione (M-IV) were determined in male C57BL/6 mice following a single oral (4.5 mg/kg) dose of either M-III or MIN-102 (5-[[4-[2-[5-(1-hydroxyethyl)pyridin-2-yl]ethoxy]phenyl]methyl]-1,3-thiazolidine-2,4-dione hydrochloride).

Animals were administered orally with a suspension formulation of either M-III or MIN-102 in 0.1% Tween 80 and 99.9% NaCMC in water (0.5% w/v) at 4.5 mg/kg dose. The blood samples were collected from a set of three mice at each time point in labeled micro centrifuge tubes containing K₂EDTA solution as anticoagulant at Pre-dose, 0.25, 0.5, 1, 2, 4, 8, 12 and 24 hr. Plasma samples were separated by centrifugation of whole blood and stored below −70±10° C. until bioanalysis. All samples were processed for analysis by protein precipitation using acetonitrile and analyzed with fit-for-purpose LC-MS/MS method (LLOQ=2.46 ng/mL for M-III and 2.49 ng/mL for M-IV). Pharmacokinetic parameters were calculated using the non-compartmental analysis tool of Phoenix WinNonlin® (Version 6.3).

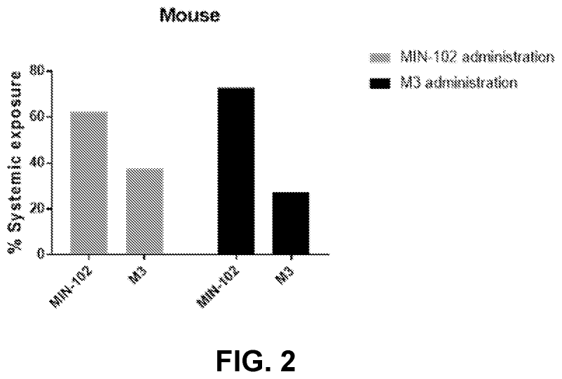

Percentages of systemic exposure (AUC) of M-III and M-IV following a single oral dose administration of either of MIN-102 or M-III to male mice (Dose: 4.5 mg/kg) are shown in FIG. 2. As can be seen in FIG. 2, M-III is metabolized to M-IV and M-IV is metabolized to M-III after oral administration. In terms of exposure (AUC), M-IV represents about 62 and 75% of the total exposure and M-III represents about 38% and 27% of the total exposure after either MIN-102 or M-III administration.

### Example 3

The plasma pharmacokinetics of 5-[[4-[2-[5-acetylpyridin-2-yl]ethoxy]phenyl]methyl]-1,3-thiazolidine-2,4-dione (M-III; M3) and 5-[[4-[2-[5-(1-hydroxyethyl)pyridin-2-yl]ethoxy]phenyl]methyl]-1,3-thiazolidine-2,4-dione (M-IV; M4) were determined in male Sprague Dawley following a single oral (10 mg/kg) dose administration of either M-III or MIN-102.

Six animals were dosed orally with a suspension formulation of either M-III or MIN-102 in 0.1% Tween 80 and 99.9% CMC (0.5% w/v solution in RO water) at 10 mg/kg.

The dosing volume administered was 10 mL/kg. Blood samples (approximately 60 μL) were collected from retro-orbital plexus of three rats at at 0.08, 0.25, 0.5, 1, 2, 4, 8, 12 and 24 hr (IV) and at predose, 0.25, 0.5, 1, 2, 4, 8, 12 and 24 hr. Samples were collected into labeled micro-tubes, containing K₂EDTA solution (20% K₂EDTA solution) as an anticoagulant. Plasma was immediately harvested from the blood by centrifugation at 4000 rpm for 10 min at 4±2° C. and stored below −70° C. until bioanalysis.

Concentrations of M-III and M-IV in rat plasma samples were determined by fit-for-purpose LC-MS/MS method. Non-Compartmental-Analysis module in Phoenix WinNonlin® (Version 6.3) was used to assess the pharmacokinetic parameters. Peak plasma concentrations (Cₐx) and time for the peak plasma concentrations (Tₘₐₓ) were the observed values. The areas under the concentration time curve (AUCₗₐₛₜ and AUC_{inf}) were calculated by linear trapezoidal rule.

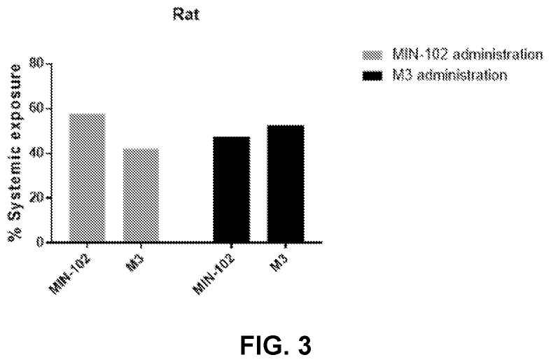

Percentages of systemic exposure (AUC) of M-III and M-IV following a single oral dose administration of either of MIN-102 or M-III to male rats (Dose: 10 mg/kg) are shown in in FIG. 3. As can be seen in FIG. 3, M-III is metabolized to M-IV and M-IV is metabolized to M-III after oral administration. In terms of exposure (AUC), M-IV represents about 58% and 48% of the total exposure and M-III represents about 42% and 53% of the total exposure after either MIN-102 or M-III administration.

### Example 4

Data from a multiple ascending dose (MAD) study of 5-[[4-[2-[5-(1-hydroxyethyl)pyridin-2-yl]ethoxy]phenyl]methyl]-1,3-thiazolidine-2,4-dione hydrochloride in human subjects at 135 mg and 270 mg confirmed that there was no change in clearance with time. The mean AUC_{0-tau ss}(% CV) (135 μg·h/mL (20) and Cmax ₛₛ(% CV) (9488 ng/ml (17) was determined at the steady state day 8 in case 135 mgrs and the mean AUC_{0-tau ss}(% CV) (299 μg·h/mL (21) and Cmax ₛₛ(% CV) (17200 ng/ml (18) was determined at the steady state day 8 in case 270 mgrs.

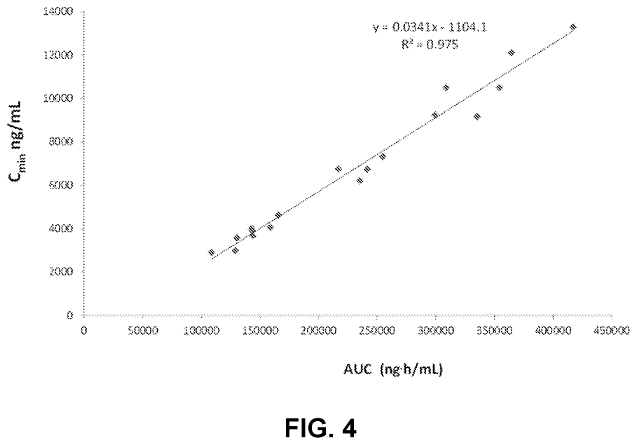

The day 8 data from the MAD study showed that C_{min ss} and AUCₛₛ were correlated as shown in FIG. 4. The equation describing the line of best fit indicates that the C_{min ss} associated with the target AUCₛₛ (200 μg·h/mL) for effect is 5716 ng/mL.

Across all ascending dose studies in humans the increase in dose is linearly related to the increase in AUC. Because of the inter subject variability in clearance it is required to select a start dose which is not excessively above the 200 μg·h/mL target AUCₛₛ.

The PK data from the MAD study was used to generate a start dose which is most likely no cause toxicity or adverse events in subjects. A dose of 150 mg was chosen as it would give a geometric mean AUCₜₐᵤ of 167 μg·h/mL SD 33 with a 95% confidence interval of 102-232 μg·h/mL. At this dose approximated 75% of patients will be below 200 μg·h/mL and it is unlikely that any patient will exceed 240 μg·h/mL.

After 2 weeks of dosing a C_{min ss} PK sample will be collected from each patient and the result will be used to adjust the dose of 5-[[4-[2-[5-(1-hydroxyethyl)pyridin-2-yl]ethoxy]phenyl]methyl]-1,3-thiazolidine-2,4-dione hydrochloride using Equation 2:

$$n e w a m o u n t in mg = 150 \times ( \frac{5716}{P C} ) , \qquad Equation 2$$

wherein PC is the plasma concentration in ng/mL of 5-[[4-[2-[5-(1-hydroxyethyl)pyridin-2-yl]ethoxy]phenyl]methyl]-1,3-thiazolidine-2,4-dione.

The dosing suspension is 15 mg 5-[[4-[2-[5-(1-hydroxyethyl)pyridin-2-yl]ethoxy]phenyl]methyl]-1,3-thiazolidine-2,4-dione hydrochloride per mL and thus the new dose will be rounded to the nearest 0.1 mL.

### Example 5

Dose calculation of 5-[[4-[2-[5-acetylpyridin-2-yl]ethoxy]phenyl]methyl]-1,3-thiazolidine-2,4-dione for humans

The suitable dose of 5-[[4-[2-[5-acetylpyridin-2-yl]ethoxy]phenyl]methyl]-1,3-thiazolidine-2,4-dione, or a pharmaceutically acceptable salt, for a human of 70 kg was extrapolated from the metabolization studies described in Example 1. After administration of 50 mg/kg of 5-[[4-[2-[5-acetylpyridin-2-yl]ethoxy]phenyl]methyl]-1,3-thiazolidine-2,4-dione to mice, the AUC_{inf} was 337 μg·h/mL.

It can be assumed that dose-exposure is linear. The minimal effective exposure of 5-[[4-[2-[5-(1-hydroxyethyl)pyridin-2-yl]ethoxy]phenyl]methyl]-1,3-thiazolidine-2,4-dione hydrochloride is 130 μg·h/mL in mice which corresponds to administration of 50 mg/kg of 5-[[4-[2-[5-(1-hydroxyethyl)pyridin-2-yl]ethoxy]phenyl]methyl]-1,3-thiazolidine-2,4-dione hydrochloride to mice. It follows that the dose to achieve this exposure in mice is 19 mg/kg.

The human equivalent dose (HED) can be calculated as follows: 19 mg/kg×0.08=1.52 mg/kg weight. For a human weighing 70 kg, the dose would be 100 mg.

### Example 6

### Synthesis of 5-[[4-[2-[5-acetylpyridin-2-yl]ethoxy]phenyl]methyl]-1,3-thiazolidine-2,4-dione

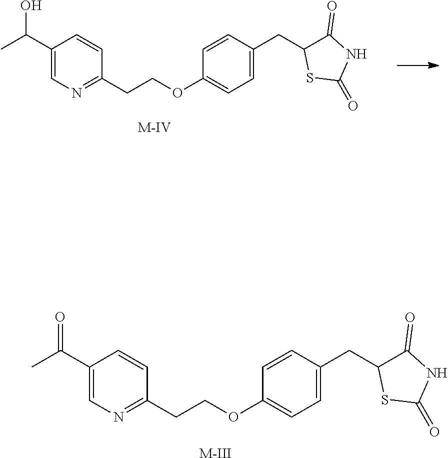

The starting compound 5-[[4-[2-[5-(1-hydroxyethyl)pyridin-2-yl]ethoxy]phenyl]methyl]-1,3-thiazolidine-2,4-dione (M-IV) can be prepared as described in WO 2015/150476 A1.

5-[[4-[2-[5-acetylpyridin-2-yl]ethoxy]phenyl]methyl]-1,3-thiazolidine-2,4-dione (M-III) was prepared from 5-[[4-[2-[5-(1-hydroxyethyl)pyridin-2-yl]ethoxy]phenyl]methyl]-1,3-thiazolidine-2,4-dione (M-IV) as follows:

PCC (pyridinium chlorochromate) (641 mg, 2.97 mmol, 2 eq.) was added to a solution of compound M-IV (554 mg, 1.49 mmol, 1.0 eq) in THE (30 mL) and the resulting mixture was stirred at room temperature for 12 h. After consumption of the starting material as determined by TLC, the reaction mixture was cooled and filtered over a celite pad. The filtrate was concentrated on rotavapor to minimum volume, and the concentrate was diluted with water (30 mL) and extracted in ethyl acetate (2×50 mL). The combined organic layer was washed with water (50 mL) followed by brine solution (50 mL), dried over anhydrous sodium sulphate, and concentrated. The crude compound was purified by flash column chromatography (dichloromethane/methanol) to afford the desired compound M-III as a white solid.

¹H NMR spectra were recorded on 400 MHz Varian NMR spectrometer using DMSO-d₆ as the solvent.

LC-MS analysis of the compounds was conducted with the following method: Column: Agilent Zorbax 3.5 μm, SB-C8 (4.6×75 mm); wavelength: 210/254 nm; flow: 1 mL/min; run time: 7 min; Time & mobile phase-gradient (time in min/B): 0/5, 3.5/90, 5/90, 5.5/5, 7/5 [B: Acetonitrile; A: Formic Acid (0.1% in water)]; MASS: Agilent-single quad-multimode-APCI-ESI.

Yield: 349 mg (63%).

ES-MS [M+H]⁺: 371.1; t_{R}=4.02 min;

¹H NMR (400 MHz, DMSO-d₆): δ 12.00 (s, 1H), 9.06 (d, J=2.0 Hz, 1H), 8.22 (dd, J=8.0, 2.4 Hz, 1H), 7.53 (d, J=8.4 Hz, 1H), 7.13 (d, J=8.4 Hz, 2H), 6.85 (d, J=8.4 Hz, 2H), 4.88-4.84 (m, 1H), 4.36 (t, J=6.4 Hz, 2H), 3.31 (m, 1H, merged with peak from H-O-D), 3.26 (t, J=6.4 Hz, 2H), 3.07-3.01 (m, 1H), 2.60 (s, 3H).

### Example 7

### Evaluation of Mitochondrial Pyruvate Carrier (MPC) Inhibitory Activity of MIN-102

### BRET-Assay

To monitor the activity of the MPC in real time, i.e., the MPC inhibitory activity (IC₅₀), a BRET assay was used transfecting the appropriate chimeric proteins in HEK cells as described in Compan et al., Molecular Cell 59:491-501 (2015).

The MPC is a heterodimer composed of two subunits, MPC1 and MPC2. MPC1 and MPC2 interact to form an active carrier. In the assay, MPC2 was fused to Rluc8 (Donor) and MPC1 to Venus (Acceptor). These chimeric proteins were stably expressed in HEK cells. BRET activity was measured following addition of coelenterazine in the culture medium. Coelenterazine enters into cells and in contact with luciferase Rluc8 emits light, which activates the emission of fluorescence by the Acceptor, provided the distance between the Donor and Acceptor is <100 nm. If the distance between Donor and Acceptor is >100 nm, no BRET activity is measured. The level of BRET activity reflects a change in the conformation of the MPC: it is high when the carrier is in a closed conformation, low when the carrier is at rest and intermediary when it transports pyruvate. In this case, the BRET activity is the mean value between the BRET value when the carrier is at rest (Maximal distance between Donor and Acceptor) and the BRET value when it is closed (Shortest distance between Donor and Acceptor)

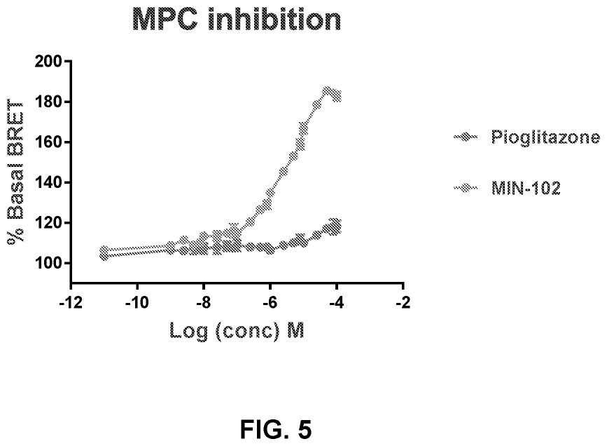

A wide range of concentrations of each of the tested compounds was used from 1 nM to 100 μM. The dose response curves of the tested compounds MIN-102 and pioglitazone are shown in FIG. 5.

The BRET activity measured for each tested compound was compared with the BRET activity obtained when HEK cells are incubated in PBS (resting state) and in PBS+pyruvate, which corresponds to the intermediary value between the resting state and the close state (maximal closure obtained with UK5099). Table 1 below provides the IC₅₀ values for the tested compounds MIN-1202, pioglitazone, rosiglitazone, and UK5099 obtained in the BRET assay described above.

TABLE 1

| Compound | IC50 | |
|---|---|---|
| MIN-102 | 4.1 | μM |
| Rosiglitazone | 2 | μM |
| UK5099 | 17 | nM |
| Pioglitazone | >100 | μM |

MIN-102 inhibits the MPC activity in the BRET assay with an IC₅₀ value of 4.1 μM. The activity of MIN-102 is slightly lower than the activity of Rosiglitazone (IC₅₀=2 μM). Accordingly, MIN-102 is a MPC inhibitor with an IC₅₀ of 4.1 μM, whereas pioglitazone does not inhibit MPC having an IC₅₀ value more than 100 μM.

### Mitochondrial Respiration

To determine whether MIN-102 has any effect on pyruvate-mediated mitochondrial respiration, the extracellular flux analyzer Seahorse was used as described in Compan et al.. Seahorse experiments were performed in the following cell lines: HeLa (Cervix cancer cells), A549 (lung cancer cells), wild type MDA MB 231, and MDA MB 231 in which MPC2 has been deleted, leading to inactivation of the MPC (MDA MB231 KO). MDA MB231 cells are epithelial breast cancer cells. Cells were incubated with increasing concentrations of the compounds for one hour at 37° C. before oxygen consumption rate (OCR) measurements. The Seahorse analyzer allowed to measure basal and maximal respiration measured upon depolarization with 1 μM CCCP. The results are shown in FIG. 4 and FIG. 5.

Effects of MIN-102 on oxygen consumption rates (OCR) in HeLa cells (FIG. 4A) and A549 cells (FIG. 4B) in a representative experiment of n=3. OCR values are expressed as ratios of OCR in the presence of different concentrations of compounds over the OCR in PBS alone. IC₅₀ in both cells lines was around 5 μM.

FIG. 5 shows the effects of MIN-102 on wild type MDA MB231 cells (FIG. 5A) and MDA MB231 KO cells (FIG. 5B). The KO cells have been deleted of the MPC2 gene and therefore they display no MPC activity. The top panel show a representative experiment of either wild type (WT) or KO cell lines. The bottom panel shows the mean values of maximal OCR in 3 different experiments. OCR values are expressed as ratios of OCR in the presence of different concentrations of the tested compound over the OCR in PBS alone.

### Conclusion

MIN-102 inhibits the MPC activity with an IC₅₀ value of 4.1 μM and inhibits oxygen consumption in a MPC dependent manner. Indeed, MIN-102 does not inhibit oxygen consumption when the activity of the MPC has been genetically deleted, supporting that MIN-102 is a specific inhibitor of MPC. The inhibitory activity of MIN-102 on the MPC is low compared to the activity of UK5099 (IC₅₀=17 nM), a potent chemical compound inhibitor of MPC, and slightly lower than, but in the same range as, the activity of rosiglitazone (IC₅₀=2 μM). MIN-102 is significantly more potent than pioglitazone.

Based on the results, it can be concluded that MIN-102 would offer a much better treatment than pioglitazone for diseases in which the energetic requirements are modified.

### Example 8

### MIN-102 Significantly Increases Adiponectin Levels in Plasma

Mitochondrial function is linked to adiponectin synthesis in adipocytes, and mitochondrial dysfunction in adipose tissue may explain decreased plasma adiponectin levels in obesity. Impaired mitochondrial function activates a series of mechanisms involving ER stress, INK, and ATF3 to decrease adiponectin synthesis. See, Eun Hee Koh et al., Diabetes 56(12):2973-2981 (2007). In addition, hepatic adiponectin receptors are diminished in NASH patients and adiponectin knockout mice develop a more extensive liver fibrosis compared with wild-type animals, whereas adenovirus-mediated overexpression of adiponectin ameliorates liver damage in wild-type mice. (See, e.g., Kamada et al., Gastroenterology 125:1796-1807 (2003)).

Evaluation of effect of MIN-102 on adiponectin was performed in Sprague Dawley wild type rats as a measure of PPAR gamma engagement. The rats were treated for 7 days with increasing doses of MIN-102 at 54 mg/Kg/day. Plasma were obtained at 1 h after the last MIN-102 administration. Adiponectin levels were measured by ELISA. Results were represented as mean+standard error of the mean of n=8. Data were analyzed by Kruskal-Wallis followed by the Dunn post-hoc test versus the vehicle group (**** p<0.0001).

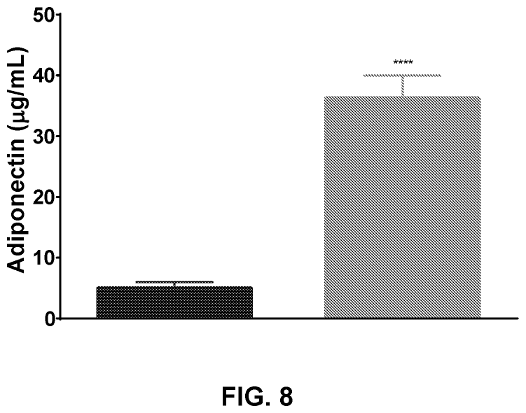

As shown in FIG. 8, MIN-102 treatment significantly increased the levels of adiponectin. Accordingly, it can be concluded based on these data that because MIN-102 treatment significantly increases the levels of adiponectin, MIN-102 could also correct the deficiency of adiponectin observed in patients suffering from a mitochondrial disease.

### Example 9

### Effects of MIN-102 in the Methionine Choline Deficient Diet Fed Mice

The preventive effects of MIN-102 was evaluated in a 7-week Methionine Choline Deficient (MCD) diet NASH mouse model (Verdelho Machado et al.). After the acclimation period, C57BL6/J male mice (n=20) were weighed and randomized into 2 homogenous treatment groups based on body weight (n=10/group), put on a MCD diet, and treated BID orally with vehicle or MIN-102 for 7 weeks.

MIN-102 was dosed 62.5 mg/kg BID orally by gavage.

When C57BL6/J mice are fed a MCD diet, they rapidly develop liver steatosis, inflammation and fibrosis with concomitant increase in plasma alanine transaminase (ALT)/aspartate aminotransferase (AST) levels.

### Material and Methods

After the acclimation period, C57BL6/J male mice (n=20) were weighed and randomized into 2 homogenous treatment groups based on body weight (n=10/group), put on a MCD diet, and treated BID orally with a vehicle or MIN-102 (125 mg/Kg/day) for 7 weeks. Body weight was measured 3 times/week until the end of the experimental phase.

At 7 weeks of diet/treatment, mice were weighed and treated at ˜08:00 am in the morning, then bled (maximal volume/EDTA) at ˜1:00 pm. Plasma was then immediately isolated and stored at −80° C. prior to assay plasma ALT and AST. The plasma volume left over was stored at −80° C. for eventual additional analysis.

After blood collection, the mice were sacrificed by cervical dislocation under isoflurane anesthesia and exsanguinated with sterile saline.

A NAFLD scoring system (NAS) adapted from Kleiner et al. (Hepatology. 41(6):1313-1321 (2005)) using the criteria described in the Table 1 below:

TABLE 2
NAFLD Scoring System (“NAS”)

| Score | Steatosis | Inflammation | Fibrosis | Hepatocyte ballooning |
|---|---|---|---|---|
| 0 | <5% of | No foci | None | None |
| | liver parenchyma | | | |
| 1 | 5-to-33% of | <2 foci at 20x field | Zone 3 and/or | Minimal to mild focal |
| | liver parenchyma | | perisinusoidal | involving fewer than 3 |
| | | | fibrosis | hepatocytes per foci |
| 2 | 34-to-66% of | 2-to-4 foci at 20x | As grade 1 and | Moderate multifocal |
| | liver parenchyma | field | portal fibrosis | involving more than 3 |
| | | | | hepatocytes per foci |
| 3 | >66% of | >4 foci at 20x field | As grade 2 and | Prominent multifocal |
| | liver parenchyma | | bridging fibrosis | involving large number |
| | | | | of hepatocytes |
| 4 | Not applicable | Not applicable | Cirrhosis | Not applicable |

Several other histopathological observations described in clinical human cases and originally reported in the NAS scoring system published by Kleiner et al. were not observed in this animal study, such as lipogranuloma, acidophil bodies, megamitochondria, and pigmented macrophages. Therefore, it was elected not to include them in the scoring system described above. An individual mouse NAS total score was calculated for each animal by summing up the score for (1) hepatocellular steatosis, (2) liver inflammation, (3) lobular fibrosis, and (4) hepatocyte ballooning.

### Results

As expected, the mice under MCD diet showed substantial body weight loss. However, the mice treated with MIN-102 showed a less severe decline in body weight loss, from day 14 to day 50, leading to significant differences between day 30 and day 50.

As also expected, MCD diet resulted in very high ALT and AST plasma levels (mean values of 480 U/L and 455 U/L, respectively) at the end of the treatment. The treatment with MIN-102 substantially reduced both plasma ALT and AST levels by 78% and 55%, respectively (both p<0.01 vs. vehicle).

The mice treated with MIN-102 did not show a change in hepatic cholesterol levels, but showed a dramatic reduction in hepatic triglycerides levels by 92% (p<0.001 vs. vehicle).

Histology analysis was performed (oil red O, H&E and Sirius Red staining) for NAFLD scoring system (NAS) for liver steatosis, inflammation, fibrosis and hepatocyte ballooning.

Mean NAS group scores were 3.40±0.3 and 0.44±01 in vehicle and MIN-102, respectively (p<0.001 vs. vehicle). The strong reduction in the NAS score was related to a blunted steatosis score (p<0.001 vs. vehicle), which was confirmed by an extremely low oil red o staining % as compared with vehicle (p<0.001), and a total disappearance of inflammation.

In conclusion, the present study demonstrates a strong reduction in liver steatosis and inflammation in MCD mice treated with MIN-102.

### Example 10

### Plasma Pharmacokinetics of 5-[[4-[2-[5-acetylpyridin-2-yl]ethoxy]phenyl]methyl]-1,3-thiazolidine-2,4-dione (M-III or M3) in Dogs

The plasma pharmacokinetics of 5-[[4-[2-[5-acetylpyridin-2-yl]ethoxy]phenyl]methyl]-1,3-thiazolidine-2,4-dione (M-III; M3) and 5-[[4-[2-[5-(1-hydroxyethyl)pyridin-2-yl]ethoxy]phenyl]methyl]-1,3-thiazolidine-2,4-dione (M-IV; M4) were determined in male Beagle dogs following a single oral (3 mg/kg) dose administration of either M3 or MIN-102.

Six animals were dosed orally with a suspension formulation of either M-III or MIN-102 in 0.1% Tween 80 and 99.9% CMC (aqueous carboxymethylcellulose 0.5% w/v solution in RO water) at 3 mg/kg. Blood samples (0.5 ml) were collected from the saphenous or cephalic or jugular veins of dogs at predose, 0.5, 1, 2, 4, 6 and 24 hr. Samples were collected into labeled micro-tubes, containing K2EDTA solution (20% K2EDTA solution) as an anticoagulant. Plasma was immediately harvested from the blood by centrifugation at 4000 rpm for 10 min and stored below −70° C. until bioanalysis.

Concentrations of M3 and M-IV in dog plasma samples were determined by fit-for-purpose LC-MS/MS method. Non-Compartmental-Analysis module in Phoenix WinNonlin® (Version 6.3) was used to assess the pharmacokinetic parameters. Peak plasma concentrations (Cmax) and time for the peak plasma concentrations (Tmax) were the 59 observed values. The areas under the concentration time curve (AUClast and AUCinf) were calculated by linear trapezoidal rule.

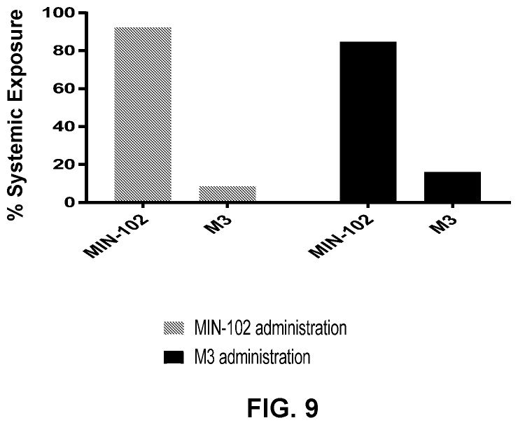

Percentages of systemic exposure (AUC) of M3 and M-IV following a single oral dose administration of either of M3 or MIN-102, respectively, to male dogs (Dose: 3 mg/kg) are shown in in FIG. 9. As can be seen in FIG. 9, M3 is metabolized to M-IV (labeled MIN-102 in the figure) and MIN-102 is metabolized to M3 after oral administration. In terms of exposure (AUC), M-IV (labeled MIN-102 in the figure) represents about 92% and 84% of the total exposure and M3 represents about 8% and 16% of the total exposure after either MIN-102 or M3 administration

### Example 11

### Examples of MDCKII Monolayer Assays

### Test System

The monolayer assays were performed using parental and MDR1 transfected MDCKII cell monolayers. MDCKII and MDCKII-MDR1 cells were cultured in Dulbecco's Modified Eagle's Medium with 4.5 g/L glucose (DMEM) supplemented with 10% (v/v) fetal bovine serum (FBS) at 37±1° C. in an atmosphere of 95:5 air:CO₂ in cell culture flasks prior to seeding into 24-transwell inserts. Transfected and parental MDCKII cells were cultured on the inserts with 400 μL medium per well on the apical side and 25 mL in a single-well receiver tray for all 24 wells on the basolateral side, for 96 hours. Medium was changed 24 hours before the experiment. Trans-epithelial electric resistance (TEER) of each well was measured to confirm the confluency of the monolayers prior to the experiments. Permeability incubations were carried out in Hank's Buffered Salt Solution (HBSS) at 37±1° C. Apical to basolateral permeability of Lucifer yellow (LY) was assessed as a low permeability control and antipyrine was as a high permeability compound. LY was also incubated in the presence of M-III (highest testing concentration, 100 μM) in order to assess the effect of M-III on the monolayer integrity. LY samples were analyzed by measuring fluorescence with the following wavelengths: excitation—485 nm emission—520 nm, while the antipyrine samples were analyzed with LC-MS/MS.

As another follow-up, MDCKII-MDR1 substrate experiment was repeated at 10 M M-III. The amounts of M-III and M-IV were determined at the end of the incubation time. Moreover, at the end of the assay, filter inserts with cells were removed and soaked in MeOH:H₂O (2:1).

### Results

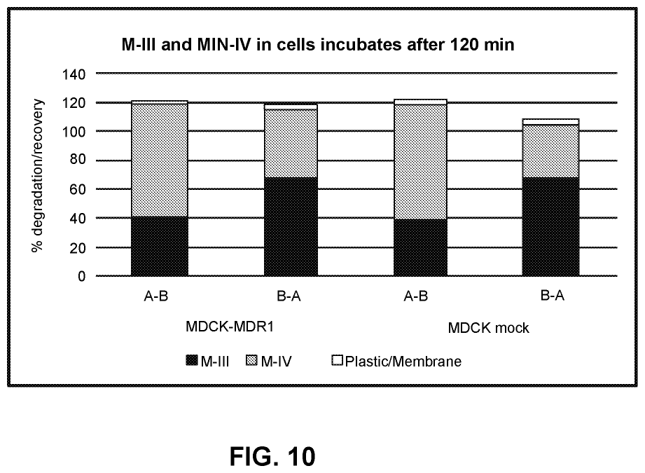

The results of the test above are depicted in FIG. 10 showing the permeability of M-III after 120 minutes of administration of M-III in both cell lines MDCKII-MDR1 and MDCKII-Mock. The recovery of M-III was poor (30-60%) due to an unexpected conversion of M-III (the main metabolite of MIN-102 or M-IV) to M-IV. The percentages of both compounds together reached the total recovery (100%±20%).

Having now fully described this disclosure, it will be understood by those of ordinary skill in the art that the same can be performed within a wide and equivalent range of conditions, formulations, and other parameters without affecting the scope of the invention or any embodiment thereof.

Other embodiments of the disclosure will be apparent to those skilled in the art from consideration of the specification and practice of the invention disclosed herein. It is intended that the specification and examples be considered exemplary only, with a true scope and spirit of the invention being indicated by the following claims.

All patents, patent applications, and publications cited herein are fully incorporated by reference herein in their entirety.

1-2. (canceled)

3. A method of treating or preventing a disease or disorder, comprising administering to a subject in need thereof a dosage form comprising an effective amount of 5-[[4-[2-[5-acetylpyridin-2-yl]ethoxy]phenyl]methyl]-1,3-thiazolidine-2,4-dione, or a pharmaceutically acceptable salt thereof, wherein said disease or disorder is selected from the group consisting of a nonalcoholic fatty liver disease, nonalcoholic steatohepatitis, a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, and a mitochondrial disease.

4-15. (canceled)

16. The method of claim 3, wherein the disease or disorder is nonalcoholic fatty liver disease (NAFLD) or nonalcoholic steatohepatitis (NASH).

17. (canceled)

18. The method of claim 3, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II, or the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus): methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal, and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

19-27. (canceled)

28. A method of treating a disease or disorder in a patient in need thereof, the method comprising administering 5-[[4-[2-[5-acetylpyridin-2-yl]ethoxy]phenyl]methyl]-1,3-thiazolidine-2,4-dione, or a pharmaceutically acceptable salt thereof, to the patient, wherein 5-[[4-[2-[5-acetylpyridin-2-yl]ethoxy]phenyl]methyl]-1,3-thiazolidine-2,4-dione is metabolized to 5-[[4-[2-[5-(1-hydroxyethyl)pyridin-2-yl]ethoxy]phenyl]methyl]-1,3-thiazolidine-2,4-dione in the patient, and wherein the 5-[[4-[2-[5-acetylpyridin-2-yl]ethoxy]phenyl]methyl]-1,3-thiazolidine-2,4-dione, or a pharmaceutically acceptable salt thereof, is administered in an amount effective to provide:
(a) a steady-state area under the curve (AUCₛₛ) of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL;
(b) a minimum steady-state plasma drug concentration (C_{min ss}) of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; or
(c) an AUCₛₛ of 5-[[4-[2-[5-(1-hydroxyethyl)pyridin-2-yl]ethoxy]phenyl]methyl]-1,3-thiazolidine-2,4-dione in plasma from the patient is about 34 μg·h/mL to about 300 μg·h/mL, and the C_{min ss} of 5-[[4-[2-[5-(1-hydroxyethyl)pyridin-2-yl]ethoxy]phenyl]methyl]-1,3-thiazolidine-2,4-dione in plasma from the patient is about 55 to about 9126 ng/mL; wherein
the AUCₛₛ of (a), the C_{min ss} of (b), or the AUCₛₛ and C_{min ss} of (c) is measured after at least five days of orally administering 5-[[4-[2-[5-acetylpyridin-2-yl]ethoxy]phenyl]methyl]-1,3-thiazolidine-2,4-dione, or a pharmaceutically acceptable salt thereof, to the patient per day.

29. The method of claim 28, wherein the AUCₛₛ of 5-[[4-[2-[5-(1-hydroxyethyl)pyridin-2-yl]ethoxy]phenyl]methyl]-1,3-thiazolidine-2,4-dione is about 100 μg·h/mL to about 300 μg·h/mL, or about 150 μg·h/mL to about 250 μg·h/mL, or about 175 μg·h/mL to about 225 μg·h/mL, or about 200 μg·h/mL, or about 30 μg·h/mL to about 100 μg·h/mL, or about 50 μg·h/mL to about 200 μg·h/mL.

30-39. (canceled)

40. The method of claim 28, wherein 5-[[4-[2-[5-acetylpyridin-2-yl]ethoxy]phenyl]methyl]-1,3-thiazolidine-2,4-dione hydrochloride is administered to the patient in need thereof.

41. The method of claim 40, wherein the 5-[[4-[2-[5-acetylpyridin-2-yl]ethoxy]phenyl]methyl]-1,3-thiazolidine-2,4-dione hydrochloride is administered to the patient as a suspension comprising about 5-15 mg of 5-[[4-[2-[5-acetylpyridin-2-yl]ethoxy]phenyl]methyl]-1,3-thiazolidine-2,4-dione hydrochloride per mL.

42. The method of claim 28, wherein the disease or disorder is selected from the group consisting of central nervous system disease or disorder, mitochondrial disease, nonalcoholic steatohepatitis (NASH), nonalcoholic fatty liver disease (NAFLD), chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, and an inflammatory respiratory disease.

43. The method of claim 42, wherein the central nervous system disorder is selected from the group consisting of a neurodegenerative disease, a cerebrovascular disease, seizure, epilepsy, a viral disease, a neuroinflammatory disease, a brain tumour, a traumatic brain injury, and a rare metabolic disease.

44. The method according to claim 42, wherein the central nervous system disorder is a neurodegenerative disease selected from the group consisting of adrenoleukodystrophy (ALD or X-ALD), amyotrophic lateral sclerosis (ALS), Parkinson's disease, multiple sclerosis, Alzheimer's disease, Huntington's chorea, Friedreich's ataxia, multiple system atrophy, progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN), neuromyelitis optica, NBIA (neurodegeneration and brain iron accumulation disorders), neuromyopathy, global or local ischemia, intracerebral haemorrhage, stroke, vascular dementia, meningitis, encephalitis, rabies, measles, mumps, poliomyelitis, herpes simplex, varicella zoster, an organic acidemia, a fatty acid disorder, and a genetic mitochondrial disorder.

45-52. (canceled)

53. The method of claim 42, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II; or a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophy, Ullrich congenital muscular dystrophy, Bethlem myopathy, oculopharyngeal distal, Emery-Dreifuss; DiGeorge syndrome; and a neuromuscular disorder selected from the group consisting of limb-girdle muscular dystrophy, inflammatory myopathies, Charcot Marie Tooth (CMT) neuropathy, and drug-induced peripheral neuropathy.

54-61. (canceled)

62. A method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising determining the plasma concentration of a PPAR-γ engagement biomarker in a sample obtained from the patient to give a baseline concentration of the PPAR-γ engagement biomarker; and
(a) administering an amount of 5-[[4-[2-[5-acetylpyridin-2-yl]ethoxy]phenyl]methyl]-1,3-thiazolidine-2,4-dione, or a pharmaceutically acceptable salt thereof, to the patient per day;
(b) obtaining a plasma sample from the patient after 5 days or more of administering according to (a);
(c) determining the plasma concentration of the PPAR-7 engagement biomarker in the plasma sample obtained in (b); and
(d) administering a recalculated amount of 5-[[4-[2-[5-acetylpyridin-2-yl]ethoxy]phenyl]methyl]-1,3-thiazolidine-2,4-dione, or a pharmaceutically acceptable salt thereof, in milligrams, based on the concentration of the PPAR-7 engagement biomarker in the plasma sample obtained in (c), wherein:
(i) an increase in the PPAR-7 engagement biomarker of about 200% or less in (c) relative to the baseline concentration of the PPAR-7 engagement biomarker comprises administering a greater amount of 5-[[4-[2-[5-acetylpyridin-2-yl]ethoxy]phenyl]methyl]-1,3-thiazolidine-2,4-dione, or a pharmaceutically acceptable salt thereof, in mg per day, to the patient;
(ii) an increase in the PPAR-7 engagement biomarker of about 600% or more in (c) relative to the baseline concentration of the PPAR-7 engagement biomarker comprises administering a lesser amount of 5-[[4-[2-[5-acetylpyridin-2-yl]ethoxy]phenyl]methyl]-1,3-thiazolidine-2,4-dione, or a pharmaceutically acceptable salt thereof, in mg per day, to the patient; and
(iii) an increase in the PPAR-7 engagement biomarker of about 200% to about 600% in (c) relative to the baseline concentration of the PPAR-7 engagement biomarker comprises administering the same amount of 5-[[4-[2-[5-acetylpyridin-2-yl]ethoxy]phenyl]methyl]-1,3-thiazolidine-2,4-dione, or a pharmaceutically acceptable salt thereof, in mg per day, to the patient.

63. The method of claim 62, wherein the PPAR-7 engagement biomarker is adiponectin.

64. A method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising:
(i) administering an amount of 5-[[4-[2-[5-acetylpyridin-2-yl]ethoxy]phenyl]methyl]-1,3-thiazolidine-2,4-dione, or a pharmaceutically acceptable salt thereof, to the patient per day;
(ii) obtaining a plasma sample from the patient after at least 4 days of administering according to (i);
(iii) determining the plasma concentration of 5-[[4-[2-[5-(1-hydroxyethyl)pyridin-2-yl]ethoxy]phenyl]methyl]-1,3-thiazolidine-2,4-dione in the plasma sample obtained in (ii); and
(iv) administering a recalculated amount of 5-[[4-[2-[5-acetylpyridin-2-yl]ethoxy]phenyl]methyl]-1,3-thiazolidine-2,4-dione, or a pharmaceutically acceptable salt thereof, in milligrams, to the patient per day as determined according to the Equation 1:
$$n e w a m o u n t in mg = S D \times ( \frac{C M T}{P C} ) , \qquad Equation 1$$
wherein:
SD is the amount of 5-[[4-[2-[5-acetylpyridin-2-yl]ethoxy]phenyl]methyl]-1,3-thiazolidine-2,4-dione, or a pharmaceutically acceptable salt thereof, administered to the patient in (i) in mg;
CMT is the C_{min target} in ng/mL of 5-[[4-[2-[5-(1-hydroxyethyl)pyridin-2-yl]ethoxy]phenyl]methyl]-1,3-thiazolidine-2,4-dione;
C_{min target}=(target AUC in ng·h/mL×0.0341±20%)−1104±20%; and
PC is the plasma concentration in ng/mL of 5-[[4-[2-[5-(1-hydroxyethyl)pyridin-2-yl]ethoxy]phenyl]methyl]-1,3-thiazolidine-2,4-dione determined in (iii).

65. A method of administering a therapeutically effective amount of 5-[[4-[2-[5-(1-hydroxyethyl)pyridin-2-yl]ethoxy]phenyl]methyl]-1,3-thiazolidine-2,4-dione to a patient in need thereof, the method comprising administering an initial dose of 5-[[4-[2-[5-acetylpyridin-2-yl]ethoxy]phenyl]methyl]-1,3-thiazolidine-2,4-dione, or a pharmaceutically acceptable salt thereof, to the patient once per day for 5 days or more; and
(a) administering a higher dose of 5-[[4-[2-[5-acetylpyridin-2-yl]ethoxy]phenyl]methyl]-1,3-thiazolidine-2,4-dione, or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is less than 149 μg·h/mL;
(b) administering a lower dose of 5-[[4-[2-[5-acetylpyridin-2-yl]ethoxy]phenyl]methyl]-1,3-thiazolidine-2,4-dione, or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is more than 241 μg·h/mL; or
(c) administering an unchanged dose of 5-[[4-[2-[5-acetylpyridin-2-yl]ethoxy]phenyl]methyl]-1,3-thiazolidine-2,4-dione, or a pharmaceutically acceptable salt thereof, to the patient once per day if the plasma concentration of 5-[[4-[2-[5-(1-hydroxyethyl)pyridin-2-yl]ethoxy]phenyl]methyl]-1,3-thiazolidine-2,4-dione in the patient is between 150 μg·h/mL and 240 μg·h/mL.

66. The method of claim 64, wherein the target AUC is about 100 μg·h/mL to about 300 μg·h/mL or about 30 μg·h/mL to about 100 μg·h/mL.

67. The method of claim 3, wherein said 5-[[4-[2-[5-acetylpyridin-2-yl]ethoxy]phenyl]methyl]-1,3-thiazolidine-2,4-dione is metabolized after oral administration to the patient providing plasma concentration of 5-[[4-[2-[5-(1-hydroxyethyl)pyridin-2-yl]ethoxy]phenyl]methyl]-1,3-thiazolidine-2,4-dione in the patient of about 30 μg·h/mL to about 300 μg·h/mL measured after at least 5 days of administering said dosage form to the patient per day.

68. the method of claim 67, wherein the plasma concentration is about 30 μg·h/mL to about 100 μg·h/mL or about 100 μg·h/mL to about 300 μg·h/mL.

69. The method of claim 3, wherein said 5-[[4-[2-[5-acetylpyridin-2-yl]ethoxy]phenyl]methyl]-1,3-thiazolidine-2,4-dione or a pharmaceutically acceptable salt is administered at a daily dose of from about 10 mg to about 500 mg.

70. The method of claim 69, wherein said 5-[[4-[2-[5-acetylpyridin-2-yl]ethoxy]phenyl]methyl]-1,3-thiazolidine-2,4-dione or a pharmaceutically acceptable salt is administered at a daily dose of from about 50 mg to about 500 mg.

71. A method of treating or preventing a central nervous system disease or disorder, comprising administering to a subject in need thereof an oral dosage form comprising an effective amount of 5-[[4-[2-[5-acetylpyridin-2-yl]ethoxy]phenyl]methyl]-1,3-thiazolidine-2,4-dione, or a pharmaceutically acceptable salt thereof, wherein said 5-[[4-[2-[5-acetylpyridin-2-yl]ethoxy]phenyl]methyl]-1,3-thiazolidine-2,4-dione is metabolized after oral administration to the patient providing plasma concentration of 5-[[4-[2-[5-(1-hydroxyethyl)pyridin-2-yl]ethoxy]phenyl]methyl]-1,3-thiazolidine-2,4-dione in the patient of about 100 μg·h/mL to about 300 μg·h/mL measured after at least 5 days of administering said oral dosage form to the patient once per day.

72. The method of claim 71, wherein the plasma concentration is about 100 μg·h/mL to about 200 μg·h/mL or about 150 μg·h/mL to about 250 μg·h/mL.

73. The method of claim 71, wherein the central nervous system disease or disorder is selected from the group consisting of a neurodegenerative disease, a cerebrovascular disease, seizure, epilepsy, a viral disease, a neuroinflammatory disease, a brain tumour, a traumatic brain injury, and a rare metabolic disease.

74. The method according to claim 71, wherein the central nervous system disease or disorder is adrenoleukodystrophy (ALD or X-ALD), amyotrophic lateral sclerosis (ALS), Parkinson's disease, multiple sclerosis, Alzheimer's disease, Huntington's chorea, Friedreich's ataxia, multiple system atrophy, progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, adrenomyeloneuropathy (AMN), neuromyelitis optica, neurodegeneration and brain iron accumulation disorder, neuromyopathy, global or local ischemia, intracerebral haemorrhage, stroke, vascular dementia, meningitis, encephalitis, rabies, measles, mumps, poliomyelitis, herpes simplex, and varicella zoster, an organic acidemia, a fatty acid disorder, or a genetic mitochondrial disorder.

75. The method according to claim 71, wherein the oral dosage form comprises from about 50 mg to about 500 mg of 5-[[4-[2-[5-acetylpyridin-2-yl]ethoxy]phenyl]methyl]-1,3-thiazolidine-2,4-dione, or a pharmaceutically acceptable salt thereof, and at least one pharmaceutically acceptable excipient.
